(19) United States
(12) Patent Application Publication
(10) Pub. No.: US 2025/0058273 A1
(43) Pub. Date: Feb. 20, 2025

(54) METHODS OF CARBON DIOXIDE REMOVAL IN FRESHWATER

(71) Applicant: CARBONRUN CARBON DIOXIDE REMOVAL LIMITED, Halifax (CA)

(72) Inventors: Shannon Sterling, Halifax (CA); Edmund A. Halfyard, Middle Sackville (CA)

(21) Appl. No.: 18/723,512

(22) PCT Filed: Dec. 23, 2022
(86) PCT No.: PCT/CA2022/051893

§ 371 Date: Jun. 24, 2024

Related U.S. Application Data
(60) Provisional application No. 63/293,689, filed on Dec. 24, 2021.
(60) Provisional application No. 63/414,191, filed on Oct. 7, 2022.

(51) Int. Cl. B01D 53/62; B01D 53/78

(52) U.S. Cl. CPC B01D 53/62; B01D 53/78; B01D 2251/30; B01D 2257/504; B01D 2258/06; B01D 2259/45

(57) ABSTRACT

Methods for carbon dioxide removal in inland waters and increased delivery of alkalinity to oceans. Methods can include adding one or more alkaline materials to one or more addition sites in an amount effective to enable enhanced weathering to occur, leading to net carbon dioxide removal from the atmosphere.

### CROSS-REFERENCE TO RELATED APPLICATIONS

The application claims priority to U.S. Provisional Application No. 63/293,689 filed on Dec. 24, 2021 and U.S. Provisional Application No. 63/414,191 filed on Oct. 7, 2022, and the entire contents of each are hereby incorporated herein by reference.

### FIELD

The present disclosure is directed to methods of carbon dioxide drawdown, also known as carbon dioxide removal (CDR). More specifically, the present disclosure relates to methods of CDR in inland waters, such as freshwater lakes and rivers.

### BACKGROUND

The following paragraphs are not an admission that anything discussed in them is prior art or part of the knowledge of persons skilled in the art.

The carbonate silicate cycle is a global biogeochemical cycle in which the dissolved inorganic carbon (DIC) produced, primarily in the form of bicarbonate (HCO₃⁻) and primarily from rock weathering at or below the land surface and is transferred along inland waters to oceans where it is held for storage over thousands/millions of years. This cycle is the long-term regulator of atmospheric CO₂ over Earth's history (Berner and Lasaga, 1989).

In natural conditions, rock weathering occurs via reactions in soil water in the lower mineral soil horizons. Soil waters have high levels of dissolved CO₂ and natural weathering reactions in soils are dominated by carbonation weathering which involve carbonic acid as a reactant.

Research on techniques for removing greenhouse gases from the atmosphere is rapidly evolving. Recent scientific reviews of the most promising and scalable CDR strategies (e.g., Fawzy et al. 2020, Terlouw et al. 2021) highlight those that employ weathering strategies such as enhanced weathering (EW) and ocean alkalinity enhancement (OAE). EW spreads large amounts of pulverized alkaline minerals onto land areas whereas OAE adds alkalinity to the ocean in coastal areas or in the open ocean (Bach et al., 2019). EW aims to increase rates of carbonation weathering of minerals and generation of alkalinity or carbonate anions, particularly bicarbonate (HCO₃⁻), that can be transferred via rivers to long-term storage in the ocean (Hamilton et al. 2007, Fawzy et al. 2020). EW work to-date has focused on croplands (Beerling et al. 2018, 2020, Kantola et al. 2017); however, it may also be applicable to forested lands.

The application of terrestrial EW on croplands and forests is challenging due to, for example, gaining access to large swaths of land and the cost of dispersion of rock dust over vast areas. Furthermore, the reactions are relatively slow and results take time to manifest (e.g., for subaerial rock weathering and forest growth, CDR can take decades). Moreover, EW assumes that the HCO₃⁻ created during weathering is either precipitated and stored within the soil or transported from soil pore water through inland waters to the ocean for storage. However, this assumption has not been widely validated and there is evidence that some conditions may lead to a breakdown of this aquatic delivery conduit (Wallin et al. 2013, Kokic et al. 2015, Horgby et al. 2019, Bertagni and Porporato 2022).

OAE seeks to increase CDR through increasing ocean alkalinity via direct applications to the ocean surface (Renforth and Henderson, 2017, Mumma et al., 2022).

Various methods for OAE strategies have been proposed. For example, U.S. Publication No. 2012/0091066 A1 discloses a method of capturing and storing excess carbon dioxide which includes seeding melt water lakes formed on glacial masses with metal hydroxides. Patent Application No. GB 2 447 513 A describes removing carbon dioxide from the atmosphere by adding powdered limestone or dolomite to the ocean. However, the dissolution reaction associated with the strategy of direct application into ocean shows slower rates, due, for example, to higher pH in oceans (Hartmann et al., 2013). U.S. Pat. No. 6,890,497 B2 discloses a method of sequestering CO₂ from a gaseous environment.

Urgent action is needed to reduce global warming to reduce the risk of severe climate change impacts, such as increased droughts, extreme rainfall events, and heatwaves (IPCC 2018, 2021). Reducing global warming depends increasingly on CDR strategies.

### INTRODUCTION

The following is intended to introduce the reader to the detailed description that follows and not to define or limit the claimed subject matter.

The present disclosure provides CDR methods, which include accelerated carbonation weathering and increased alkalinity in inland waters, such as freshwater rivers, wetlands, and lakes. These methods can draw down carbon from the atmosphere while increasing inland waters' alkalinity and ability to store carbon, locking much of the carbon dioxide into more stable bicarbonate form and delivering it to the ocean for long-term storage. The methods can increase CDR, thereby contributing to the reduction of global warming.

Accordingly, the present disclosure includes methods of removing atmospheric carbon dioxide (CO₂), including: identifying a target inland waterbody having connectivity to an ocean; selecting one or more alkaline materials to be used; locating one or more addition sites in the target inland waterbody; establishing pre-treatment alkalinity status at the one or more addition sites in the target inland waterbody to determine one or more methods of adding the one or more alkaline materials at the one or more addition sites; and adding the one or more alkaline materials to the one or more addition sites in the target inland waterbody.

Other features and advantages of the present disclosure will become apparent from the following detailed description. It should be understood, however, that the detailed description and the specific examples, while indicating embodiments of the application, are given byway of illustration only and the scope of the claims should not be limited by these embodiments but should be given the broadest interpretation consistent with the description as a whole.

### DRAWINGS

The embodiments of the application will now be described in greater detail with reference to the drawings, in which:

FIG. 1A shows a flow chart of a method of removing atmospheric carbon dioxide according to exemplary embodiments of the application.

FIG. 1B shows a schematic view of an exemplary target inland waterbody and waterbody terms used in the method of the present application.

FIG. 1C shows a schematic view of connectivity types of the target inland waterbody to the ocean as a function of alkalinity status of the drainage network of the addition site.

FIG. 1D shows a schematic view of alkaline amendment application methods and infrastructure; top left) a doser; top right) a reactor; and bottom) a barge.

FIG. 1E shows a schematic view of the locations of monitoring sites as a function of alkalinity status.

FIG. 2 shows an exemplary watershed with two alkaline material addition sites and a control and treatment monitoring site network.

FIG. 3 shows a flowchart of an exemplary process to loop carbon variable and stream chemistry data back to refine the dosing algorithm for target inland waterbody lime dosers.

FIG. 4 shows an exemplary flowchart of a method of calculating the amount of CO₂ removed from the atmosphere following the addition of the alkaline material to the target inland waterbody.

FIG. 5 shows a flowchart of an exemplary method of quantifying ecological co-benefits of the alkalinity addition.

FIG. 6 shows maps of sampling site locations in Nova Scotia, Canada in exemplary embodiments of the application. KRC is a control monitoring site, directly upstream of the alkaline addition site. KRT is a treatment monitoring site at the downstream end of the weathering zone. AF_WR is a treatment monitoring site at the river mouth.

FIG. 7 shows schematics for the design of continuous monitoring stations of A) a control site (KCR) and B) a treatment site downstream of the weathering zone (KRT) for the Killag River sites in exemplary embodiments of the application. pH, temperature, and conductivity were measured by a sonde, and CO₂ concentration was measured by an eosGP.

FIG. 8. shows measurements of HCO₃ concentrations versus distance at upstream control sites (7UKR, 9UWR, KRC), and at downstream treatment sites (KRT, KRB, BD_WR, X1WR, and AF_WR) in exemplary embodiments of the application (Case A Step F following the method in FIG. 1A). HCO₃ concentrations are derived from field measurements of CO₂ concentration and pH and the program CO₂SYS (Lewis and Wallace, 1998). Sites are as situated in FIG. 6.

FIG. 9 shows locations of a 2021 survey of short (30 min) carbon dioxide flux and pH measurements in the broader region of Nova Scotia. These measurements identify high CO₂ evasion flux levels and can be used to determine the potential for the method of the present application in new target inland waterbodies. Average CO₂ flux is shown for each site in μmol m⁻²s⁻¹.

FIG. 10 shows CO₂ flux for continuous monitoring permanent stations from June to December, 2021 of A) Killag River: KRC=control site; KRT=treatment site at the bottom of a weathering zone and B) West River in exemplary embodiments of the application: WRC=control site; WRT=treatment site at the bottom of a weathering zone.

FIG. 11 shows a relationship between CO₂ flux and pH at continuous monitoring permanent stations from Jun. to Dec. 1, 2021 of A) Killag River: KRC=control site; KRT=treatment site and B) West River in exemplary embodiments of the application: WRC=control site; WRT=treatment site.

FIG. 12 shows a relationship between CO₂ flux and water temperature at continuous monitoring permanent stations from June to December, 2021 of A) Killag River: KRC=control site; KRT=treatment site and B) West River in exemplary embodiments of the application: WRC=control site; WRT=treatment site.

FIG. 13 shows CO₂ concentration for continuous monitoring permanent stations from June to December, 2021 of A) Killag River: KRC=control site; KRT=treatment site and B) West River in exemplary embodiments of the application: WRC=control site; WRT=treatment site.

FIG. 14 shows a relationship between CO₂ concentration and pH at monitoring stations from June to December, 2021 of A) Killag River: KRC=control site; KRT=treatment site and B) West River in exemplary embodiments of the application: WRC=control site; WRT=treatment site.

FIG. 15 shows a relationship between CO₂ concentration and water temperature at stations in exemplary embodiments of the application from June to December, 2021 of A) Killag River: KRC=control site; KRT=treatment site and B) West River: WRC=control site; WRT=treatment site.

FIG. 16 shows CO₂ flux at locations in the broader region of Nova Scotia of A) sites with longer-term deployments (over 12 hours) and of B) sites with shorter term deployments (over 30 minutes).

FIG. 17 shows a relationship between CO₂ flux and pH at all spot check locations in the broader region of Nova Scotia. These measurements identify high CO₂ evasion flux levels and can be used to determine the potential for the method of the present application in new target inland waterbodies.

FIG. 18A shows the CDR calculation for Case A, for the initial alkaline amendment application method (following Step F in FIG. 1A). Here the CDR is CDR_gross, not including grey emissions.

FIG. 18B shows the CDR calculation for Case A, for the modified alkaline amendment application method (following Step H in FIG. 1A). Here the CDR is CDR_gross, not including grey emissions.

FIG. 18C shows the CDR calculation for Case B, for the initial and final alkaline amendment application method (following Step F in FIG. 1A). Here the CDR is CDR_gross, not including grey emissions.

FIG. 19 shows a schematic view of carbon removal mechanisms for the CDR of the method of the present application.

FIG. 20 shows and example of types of models used to generate baseline scenario values for Case B. FIG. 20A shows the model established between the control and treatment sites based on data collected pre-treatment.

FIG. 20B shows the projection of baseline values based on post-treatment control data using the model established in the pre-treatment stage.

### DESCRIPTION OF VARIOUS EMBODIMENTS

Various apparatuses or methods will be described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below, or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim or dedicate to the public any such invention by its disclosure in this document.

### Definitions

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this disclosure, the singular forms “a”, “an” and “the” include plural references unless the content clearly dictates otherwise.

The term “alkalinity” as used herein refers to the buffering capacity of water. More specifically, alkalinity is the excess of proton acceptors (bases) in a solution (Middelburg et al., 2020). It is a measure of the ability of the water body to neutralize acids and bases and thus maintain a stable pH level.

The term “carbon dioxide removal” (CDR) as used herein refers to is a process in which carbon dioxide gas (CO₂) is removed from the atmosphere and sequestered (for example for long period of time), resulting in net CO₂ removal from the atmosphere. CDR may refer to the strategies used to achieve CDR for example to reduce climate change impacts, and to the amounts of CO₂ that has been removed from the atmosphere and delivered to long-term storage in the ocean.

The term “carbonation weathering” as used herein refers to the weathering reactions in which H₂CO₃* is a reactant and which result in carbon drawdown and generation of alkalinity or carbonate anions, for example HCO₃⁻, as a product.

The term “CO₂ drawdown” or “CO₂ removal” or CDR as used herein refers to a process that removes CO₂ from the atmosphere. The CO₂ drawdown/removal can include short term or long-term storage. CDR_gross refers to carbon dioxide removal amounts that does not consider grey emissions (G). CDR_net refers to carbon dioxide removal amounts from which grey emissions are subtracted (i.e., CDR_gross−G=CDR_net).

The term “carbonic acid” as used herein comprises the two carbon species of CO_{2(aq)} and H₂CO₃, due to the fast reaction kinetics of CO₂ in water, and is represented herein as H₂CO₃*.

The term “strong acid” as used herein comprises acids that have a pKa value less than that of carbonic acid. The pKa of strong acids is most often less than −1.74, and includes but is not limited to sulphuric acid, nitric acid, hydrochloric acid, and organic acids such as those found in dissolved organic carbon.

The term “enhanced weathering” (EW) as used herein refers to a process that increases carbonation weathering rates through, for example, the addition of alkaline materials to land, freshwaters or oceans, and/or exposure of alkaline minerals to chemical/microbial conditions that increase carbonation weathering rates.

The term “dissolved inorganic carbon” (DIC) as used herein refers to the sum of the inorganic carbon species and is comprised of compounds such as carbon dioxide (CO₂), carbonic acid (H₂CO₃), bicarbonate (HCO₃⁻) and carbonate (CO₃²⁻).

The term “organic carbon” (OC) as used herein refers to organic compounds in water, either in dissolved or particulate form. Dissolved organic carbon (DOC) is operationally defined as OC that passes through a small mesh (e.g., 0.22 to 0.7 um).

The term “pCO₂” as used herein refers to the partial pressure of CO₂ in a fluid (i.e., in a gas or liquid).

The term “target inland waterbody” as used herein refers to an inland waterbody that is selected for removing atmospheric carbon dioxide (CO₂) according to the method of the present application.

The term “inland waters” as used herein refers to the hydrologic flow pathways comprising surface waters, ponds, streams, rivers, wetlands, lakes and estuaries that connect the land surface to the oceans.

The term “inland waterbody” is a segment of inland waters.

The term “doser” as used herein refers to a structure that both stores and disperses materials into environments, including watersheds and inland waters.

The term “bathymetric data” as used herein refers to information about the depths, volumes and shapes of underwater terrain, which may include stratification and turnover regimes.

The term “limnological characterizations” as used herein refers to aspects of the biological, chemical, physical, and geological characteristics and functions of inland waters, including discharge rate.

The term “stratification” as used herein refers to a change in the temperature at different depths in a body of water and is due to the change in water's density with gradients in temperature and salinity.

The term “discharge rate” as used herein refers to the quantity of a fluid passing through a cross-sectional area per unit time. Discharge rate can be expressed as volume per time, e.g., m³s⁻¹, and as length per time, e.g., m yr⁻¹.

The term “connectivity to an ocean” as used herein means that water from the target watershed or target inland waterbody eventually drains or flows into an ocean (directly or indirectly connected to an ocean).

The term “headwater” as used herein refers to smaller water courses at higher elevations in a watershed, as opposed to the downstream larger water courses at lower elevations in the watershed.

The term “background condition” as used herein refers to conditions that are projected to occur at treatment sites had the treatment not taken place.

The term “monitoring site” as used herein refers to a location along an inland waterway where data are collected to quantify values of carbon variables, water variables and/or ecological variables.

The term “treatment site” as used herein refers to a monitoring site downstream of the alkaline addition location.

The term “control site” as used herein refers to a monitoring site not affected by the treatment, for example, upstream of the addition site, or in neighbouring watersheds.

The term “dosing algorithm” as used herein refers to a formula or method that specifies the amount of alkaline material to be added to the target inland waterbody per unit of time.

The term “addition site” as used herein refers to a location where alkaline material is added to an inland waterbody.

The term “carbon variables” as used herein refers to the variables that represent amounts and flows of inorganic carbon in inland waters and the chemical states that determine the amounts and flows, which include alkalinity, DIC, particulate inorganic carbon, HCO₃⁻ (bicarbonate), H₂CO₃ (carbonic acid), H₂CO₃*, CO₃²⁻ (carbonate), dissolved CO₂, pCO₂, ionic strength, the carbonate system, temperature, pH, salinity and pressure.

The term “water variables” as used herein refers to the variables that represent the water quality and flows in inland waters and the chemical states that determine the amounts and flows, which include DOC, particulate organic carbon, anions, cations, metals, isotopes, microbial composition and diversity, eDNA, river discharge, Reynolds number, turbidity, conductivity, specific conductivity, dissolved oxygen, and river velocity.

The term “ecological variables” as used herein refers to the variables that represent the health of ecosystems and may refer to species population and productivity, indicators of stress in organisms, presence of invasive species or toxic microbial/algal blooms, nutrient concentrations, heavy metal and toxic substance concentrations, or biodiversity.

The term “watershed” as used herein refers to an area of land that channels rainfall and snowmelt through groundwater and inland waters that eventually flow to a defined point of interest. The watershed for a defined point of interest is the area of land that drains water to the point of interest.

The term “drainage network” as used herein refers to all inland waters that drain towards the point of interest of a watershed.

The term “river mouth” as used herein refers to a section of a river in it lowermost reach just above where it enters the ocean or a large alkaline lake. In cases where it enters the ocean, the “river mouth” may include the zone that extends through the tidal zone to the estuary.

The term “alkaline material” as used herein refers to a type of substance that when dissolved produces a pH higher than 6.0.

The term “point of interest” as used herein refers to the most downstream point of a watershed.

The term “grey emissions” as used herein refers to CO₂ emissions created from the creation and delivery of the alkaline amendment to the addition site, and to CO₂ emissions created during the alkaline amendment addition process.

### Methods of the Present Disclosure

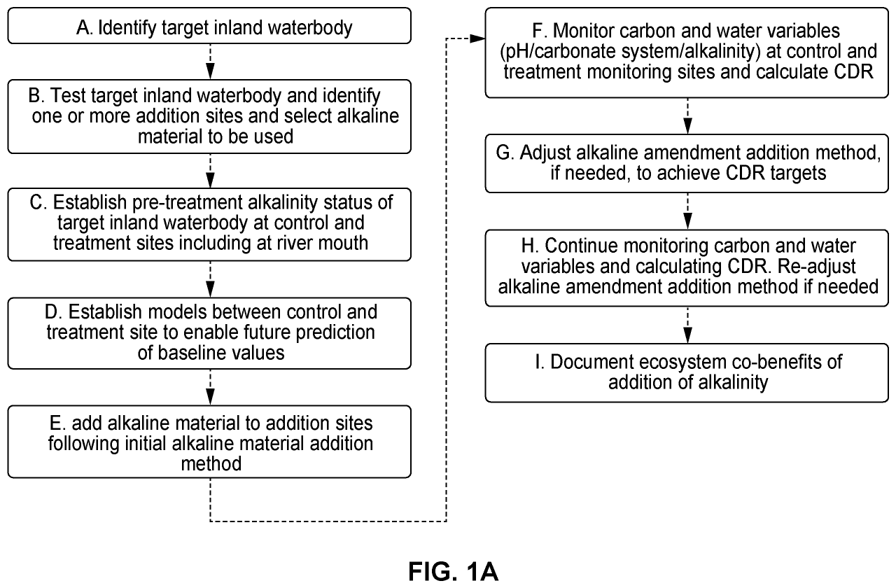

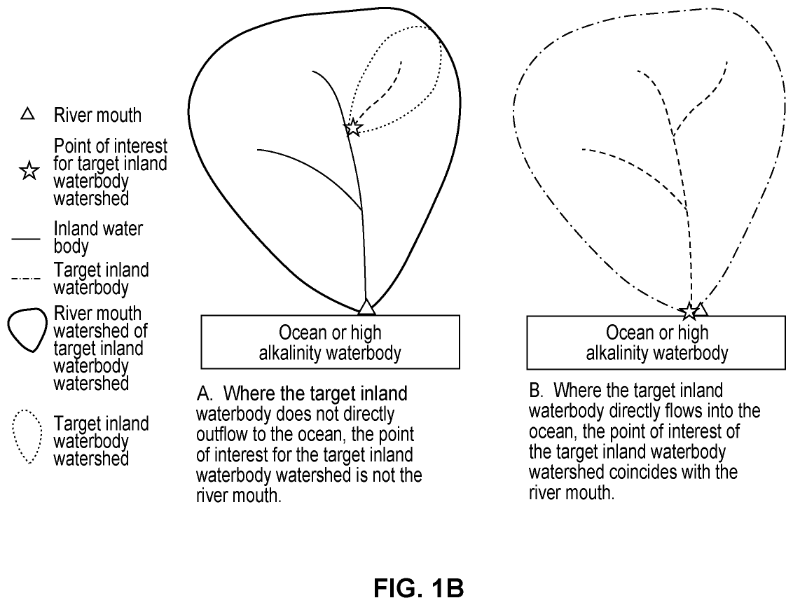

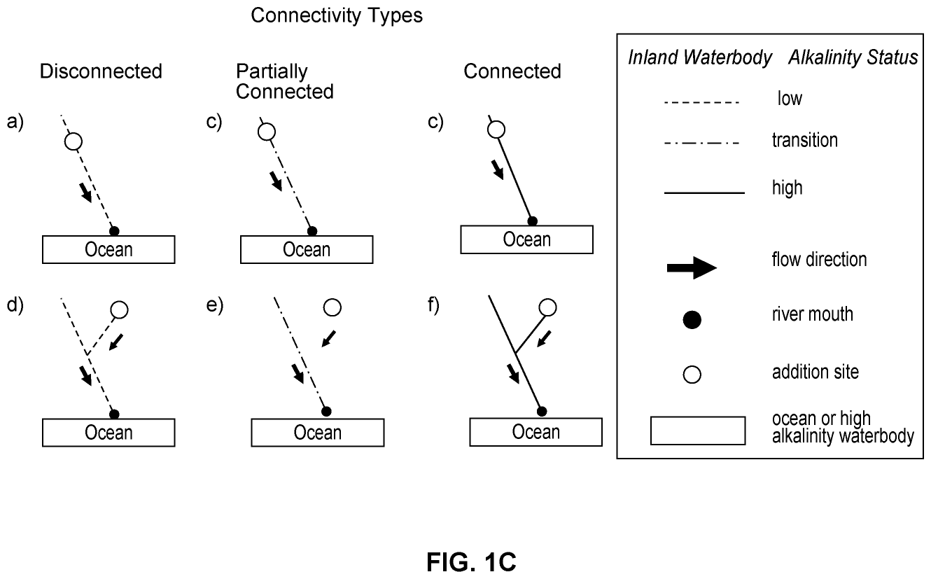

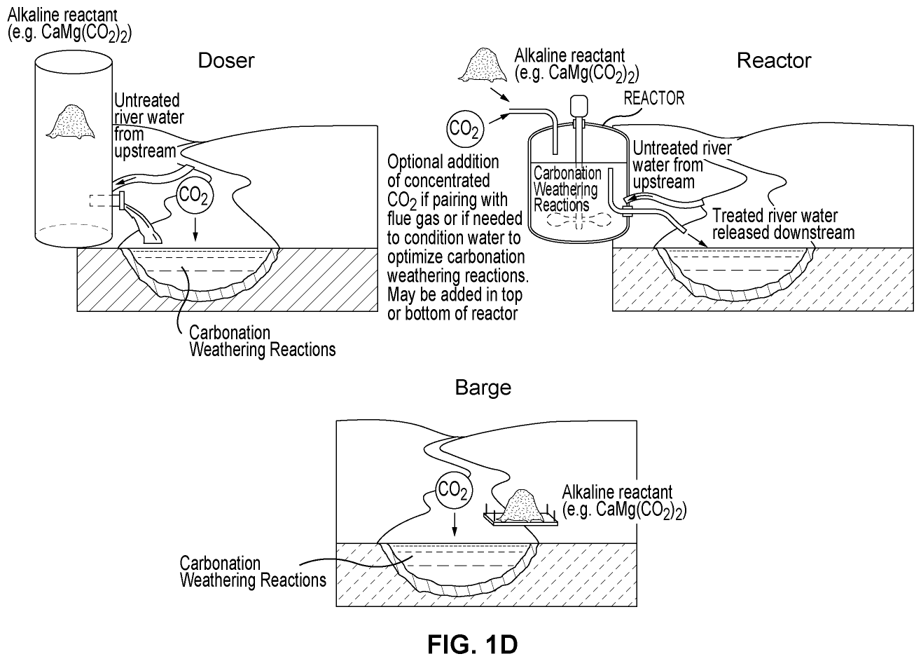

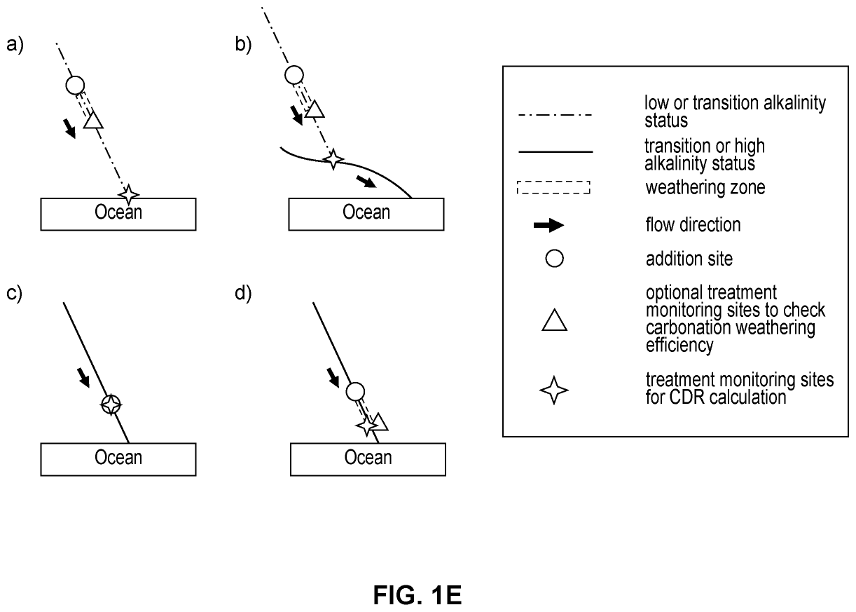

The present disclosure includes methods of removing atmospheric carbon dioxide (CO₂) from the atmosphere (FIG. 1A), including:
identifying a target inland waterbody having connectivity to an ocean; selecting one or more alkaline materials to be used;
locating one or more addition sites in the target inland waterbody;
establishing pre-treatment alkalinity status at the one or more addition sites in the target inland waterbody to determine one or more methods of adding the one or more alkaline materials at the one or more addition sites; and
adding the one or more alkaline materials to the one or more addition sites in the target inland waterbody.

In some embodiments, the identified target inland waterbody is directly or indirectly connected to an ocean. In some embodiments, the identified target inland waterbody is directly connected to an ocean. In some embodiments, the identified target inland waterbody is indirectly connected to an ocean. In some embodiments, the target inland waterbody is a pond, a stream, a lake, a river, a man-made channel or conduit, an estuary, or a wetland. In some embodiments, the target inland waterbody is a river.

In some embodiments, the step of identifying the target inland waterbody (Step A in FIG. 1A) includes gathering information on the target inland waterbody watershed properties. In some embodiments, the step of identifying the target inland waterbody includes gathering information on at least one of hydrology, flow pathways, flow velocity, discharge rate, watershed boundaries, at least one carbon variable, at least one water variable, and at least one ecological variable of the target inland waterbody. In some embodiments, the step of identifying the target inland waterbody includes gathering information on at least one carbon variable of the target inland waterbody. In some embodiments, the step of identifying the target inland waterbody includes gathering information on at least one ecological variable of the target inland waterbody. In some embodiments, the step of identifying the target inland waterbody includes gathering information on at least one ecological variable of the target inland waterbody and/or of the ocean.

In some embodiments, other factors that are taken into consideration when identifying a target inland waterbody include, but are not limited to, accessibility along the drainage network, favorable water chemistry for carbonation weathering reactions, organic carbon concentrations, fluvial geomorphology, discharge rates and/or stability of discharge regime.

In some embodiments, the identified target inland waterbody has accessibility to vehicles, and/or boats along some or part of the drainage network along the drainage network. In some embodiments, the identified target inland waterbody has hydrological discharge rate and coefficient of variation suitable for the addition of alkalinity. In some embodiments, the identified target inland waterbody has favorable water chemistry for carbonation weathering reactions.

In some embodiments, the target inland waterbody comprises at least one of dissolved organic carbon (DOC) concentration of less than about 30 mg/L, a discharge rate of greater than about 1 m³/s or a stable discharge regime, mean pH of about 4 to about 10, water temperature of about 0° C. to about 45° C., pCO₂ of above about 200 ppm, HCO₃ concentration below 6 mM or more than about 0.6 mM, Ca²⁺ concentration of less than about 100 mg/L or more than 5 mg/L, Mg²⁺ concentration of less than about 40 mg/L, conductivity of about less than about 1000 uS/cm, CaCO₃ concentration of less than about 1000 mg/L, specific conductivity of more than 50 uS/cm, suspended sediment concentration of less than about 1000 mg/L, a suspended sediment concentration of more than 1000 mg/L, dissolved oxygen of about 1 mg/L to about 50 mg/L, or any combination thereof.

In some embodiments, the target inland waterbody comprises a DOC concentration of about 10 mg/L to about 30 mg/L, a temperature of about 0° C. to about 45° C., mean pH of about 4.5 to about 6.3 or about 6.3 to about 9.0, a pCO₂ of above about 1000 ppm; a Ca²⁺ concentration of less than about 10 mg/L or more than about 5 mg/L, a Mg²⁺ concentration of less than about 5 mg/L, a specific conductivity of less than about 50 uS/cm or more than 50 uS/cm, a CaCO₃ concentration of less than about 10 mg/L, HCO₃⁻ concentration of about 0 to about 1 mM or more than about 0.6 mM, a suspended sediment concentration of less than about 50 mg/L, or greater than 50 mg/L, a dissolved oxygen of about 7 mg/L to about 20 mg/L, or any combination thereof.

In some embodiments, the target inland waterbody comprises at least one of a temperature of about 0° C. to about 45° C., mean pH of about 6.3 to about 9.0, a Ca²⁺ concentration of more than about 5 mg/L, HCO₃⁻ concentration of more than about 0.6 mM, a conductivity of more than about 50 uS/cm, or any combination thereof.

In some embodiments, the target inland waterbody comprises at least one of Reynold's number of greater than about 2300, discharge rate of greater than about 1 m³/s, or temperature of less than about 45° C.

In some embodiments, other factors that are taken into consideration when identifying a target inland waterbody include, but are not limited to, social context, distance to sources of the alkaline material, presence of other carbon dioxide removal or enhanced weathering projects in the watershed, presence of anthropogenic point source inputs to the target inland waterbody, importance and/or title for indigenous peoples, and transportation network.

In some embodiments, the target inland waterbody is identified based on at least one of a distance to sources of the one or more alkaline materials, a distance to industrial infrastructure, a distance to logistical resources and presence of an existing land-based enhanced weathering project. In some embodiments, the industrial infrastructure includes, but is not limited to, flue gas sources or a dam. In some embodiments, the target inland waterbody is identified based on, at least one of, to minimize the distance to sources of the one or more alkaline materials, to minimize the distance to industrial infrastructure, to minimize the distance to logistical resources and availability to connect to the existing or future land-based enhanced weathering projects.

In some embodiments, the target inland waterbody includes the entire drainage network (a drainage network) of the target inland waterbody watershed. In some embodiments, the target inland waterbody includes a segment of the drainage network of the target inland waterbody watershed (FIG. 1B). In some embodiments, the segment of the drainage network is located between headwaters and a point of interest of the target inland waterbody watershed. The size of the segment of the drainage network can be varied and is within the consideration of the person skilled in the art.

In some embodiments, the step of identifying the target inland waterbody includes testing at least one carbon variable and at least one water variable in the target inland waterbody. In some embodiments, the step of identifying the target inland waterbody includes testing pH and bicarbonate concentration. In some embodiments, the step of identifying the target inland waterbody includes testing pH and at least one of pCO₂ or CO₂ concentration.

In some embodiments, the step of identifying the target inland waterbody includes testing at least two carbon variables and at least one water variable in the target inland waterbody. In some embodiments one of the two carbon variables is pH. In some embodiments, one of the two carbon variables is bicarbonate concentration. In some embodiments, a first carbon variable of the at least two carbon variables is pH and a second carbon variable is pCO₂. In some embodiments, the first carbon variable is pH and the second carbon variable is CO₂ concentration.

In some embodiments, the target inland waterbody is acidic, with a mean pH of less than about 7.0, prior to adding the alkaline material. In some embodiments, the target inland waterbody is acidic, with a mean pH of less than about 6, prior to adding the alkaline material.

In some embodiments, the target inland waterbody is not acidic prior to adding the alkaline material. In some embodiments, the target inland waterbody is not acidic with a mean pH of above about 6.3, prior to adding the alkaline material. In some embodiments, the target inland waterbody is not acidic with a mean pH of above about 7.0, prior to adding the alkaline material. In some embodiments, the mean pH is determined by taking the average of one or more annual, bi-annual, monthly, weekly, daily, hourly, or continuous measurements of the pH.

In some embodiments, the target inland waterbody is not acidic prior to adding the alkaline material. In some embodiments, the target inland waterbody is not acidic prior to adding the alkaline material, with bicarbonate concentrations of the target inland waterbody at the river mouth below carbonate saturation concentrations. In some embodiments, the target inland waterbody is not acidic prior to adding the alkaline material, with the bicarbonate concentrations at the river mouth of the target inland waterbody between 0 and about 6000 μmol/L.

In some embodiments, the target inland waterbody is not acidic prior to adding the alkaline material based on the measurements of the mean pH, alkalinity, and/or at least one carbon variable. In some embodiments, the target inland waterbody is not acidic prior to adding the alkaline material, with bicarbonate concentrations of the target inland waterbody at the river mouth are below saturation level or between 0 and about 6000 μmol/L and the mean pH is below about 7.0 or is above about 7.0, or the mean pH is above about 6.0 or above about 6.3.

In some embodiments, the step of selecting one or more alkaline materials (Step B in FIG. 1A) includes selecting one or more alkaline materials from: carbonate rocks comprising minerals such as dolomite, or calcite; igneous/metamorphic rocks rich in alkaline earth metals, such as basalt, containing such minerals as wollastonite, calcium-rich plagioclase feldspar, olivine, and calcium and magnesium-rich pyroxenes; industrially-produced alkaline material, such as cement kiln dust, magnesium or calcium oxides; industrial materials containing alkali metals or alkaline earth metals; or any combination thereof. In some embodiments, the alkaline material is crushed dolomite.

In some embodiments, the alkaline material to be used is selected based on at least one of the following characteristics/considerations: the proximity to the source of the one or more alkaline materials, local availability, the cost of the one or more alkaline materials, an opportunity to reuse waste materials, the absence of impurities, toxins, heavy metals or other undesirable attributes, the ability to provide ecosystem co-benefits, such as the addition of calcium and magnesium in inland waters deficient in these elements, ability to optimize the introduction of alkalinity and increased pH when dissolved in freshwater, ability to optimize the speed of reaction within acceptable environmental limits. Considering local availability, the type of alkaline material is also chosen based on the following: to introduce of alkalinity and increase pH when dissolved in freshwater; to maintain a fast speed of reaction within acceptable environmental limits; to have full dissolution to avoid increased turbidity; and to optimize ecological co-benefits, such as via the introduction of base cations such as calcium. In cases where strong acids are present in the target inland waterbody, use of carbonate-based alkaline amendments needs to be carefully monitored to check for CO₂ losses due to strong acid weathering and/or low pH. An example of a method to address the potential loss of CO₂ from the application of carbonate minerals to inland waters with higher levels of strong acids is to 1) pre-treat the water so that the strong acids have been removed before the addition of carbonate minerals, either upstream or in a one- or two-stage reactor, or 2) add the carbonate minerals in a reactor where the CO₂ produced from strong acid weathering with the carbonate minerals is not released to the atmosphere, and/or rediverted into weathering of new carbonate minerals.

Method to safely deploy silicate minerals and other materials that may produce clays during dissolution or carbonation weathering, e.g., deploying silicates in impoundments or lakes where clays can settle out, so that increased turbidity downstream is prevented; an example of a method to address this is through the introduction of a reactor step to the process, where the clays settle out before the treated water is introduced into the inland waterbody.

Method to safely deploy alkaline materials, such as silicate minerals and cement kiln dust (CKD) that may release toxic metals during dissolution or carbonation weathering, e.g., adding the alkaline materials first in a reactor, where the alkaline material is dissolved in a reactor and contaminants are removed through microbial or chemical processes, until the contaminants are reduced to acceptable levels in the treated water when it is ready to be dispersed into the inland waterbody.

In some embodiments, chemical properties, weathering reactions, physics and/or kinetics of the one or more alkaline materials to be used in the target inland waterbody are determined using any methods known to the person skilled in the art.

In some embodiments, a median grain size of the one or more alkaline materials is between about 0.02 μm to about 120 mm. In some embodiments, the median grain size of the one or more alkaline materials is between about 0.03 μm to about 100 mm. In some embodiments, the median grain size of the one or more alkaline materials is between about 0.05 μm to about 80 mm. In some embodiments, the median grain size of the one or more alkaline materials is between about 0.06 μm (clay) to about 64 mm (coarse gravel).

In some embodiments, the method further includes adding microbial communities to the one or more alkaline materials, a reactor, or directly to the target inland waterbody. In some embodiments, the addition of the microbial communities to the one or more alkaline materials, a reactor, or the target inland waterbody promotes and accelerates carbonation weathering reactions (Uroz et al., 2009).

In some embodiments, the method further comprises adjusting the composition and grainsize of the one or more alkaline materials. In some embodiments, the composition of the one or more alkaline materials is adjusted via the addition of substances prior to distribution or by sourcing alternative one or more alkaline materials. In some embodiments, the grainsize of alkaline materials is adjusted in response to iterative measurements of weathering rates where the smaller grain size is used to increase weathering rates. In some embodiments, the grainsize is optimized for weathering vs the need to grind to finer grainsizes, based on individual site conditions. In some embodiments, said adjustments and optimizations avoid the deposition of the one or more alkaline materials at the bottom of the target inland waterbody and keep sediment entrained until the weathering reaction is complete, while minimizing the costs of grinding. Sediment deposition and entrainment thresholds are site-specific, and are a function of one of the following: water velocity, friction (bed material grain size), straightness of channel/fluvial geomorphology, and/or channel slope (and thus stream power/shear stress conditions in the mixing zone).

In some embodiments, the locating one or more addition sites in the target inland waterbody includes testing target inland waterbody. In some embodiments, testing target inland waterbody includes testing at least one of water slope, water discharge, anthropogenic impacts, drainage network configuration, bedrock type, ecology, species at risk or any combination thereof.

In some embodiments, the method further includes locating one addition site in the target inland waterbody. In some embodiments, the method includes locating two or more addition sites in the target inland waterbody. In some embodiments, two addition sites are located in the target inland waterbody. The selected number of addition sites is based on the size of the segment of the target inland waterbody, the waterbody's watershed properties, hydrology, pathways, flow, discharge, carbon variables, water variables, ecological variables and the like.

In some embodiments, the one or more addition sites are located adjacent to a dam. In some embodiments, the one or more addition sites are located within a dam. In some embodiments, the one or more addition sites are located above a dam. In some embodiments, the one or more addition sites are located below a dam.

In some embodiments, the one or more addition sites are located adjacent to an industrial infrastructure. In some embodiments, the one or more addition sites are located within an industrial infrastructure. In some embodiments, the one or more addition sites are located near an industrial infrastructure. In some embodiments, the industrial infrastructure is a storage silo or a reactor. In some embodiments, the industrial infrastructure is a mine, a quarry or a cement plant. As such, in some embodiments, industrial infrastructure is selected from a storage silo, a reactor, a mine, a quarry and a cement plant. In some embodiments, the industrial infrastructure produces CO₂ as a byproduct or a waste product.

In some embodiments, the one or more addition sites have turbulent waters immediately downstream of the one or more addition sites. In this embodiment, the speed and efficiency of the method of the present disclosure is increased due to physical agitation and chemical disequilibrium induced by turbulent stream flow.

In some embodiments, the one or more addition sites are located in the headwaters of the target inland waterbody. In some embodiments, the one or more addition sites are located above the river mouth of the target inland waterbody.

In some embodiments, the pre-treatment alkalinity status (Step C in FIG. 1A) is established at the one or more addition sites. In some embodiments, the pre-treatment alkalinity status is established at the one or more monitoring sites. In some embodiments, the pre-treatment alkalinity status (Step C in FIG. 1A) at the one or more addition sites in the target inland waterbody is obtained by direct measurement of the target inland waterbody using any method known in the art. In some embodiments, the pre-treatment alkalinity status is obtained by modelling of the target inland waterbody using any method known in the art. In some embodiments, the pre-treatment alkalinity status is obtained by direct measurement of the target inland waterbody and modelling of the target inland waterbody. In some embodiments, the pre-treatment alkalinity status is obtained from an existing land-based enhanced weathering project. In some embodiments, the pre-treatment alkalinity status is obtained from a separate monitoring program or project.

In some embodiments, the pre-treatment alkalinity status is determined based on at least one of alkalinity, carbon variable, strong acids and pH values of the inland waterbodies that form the drainage network from the one or more addition sites to the estuary river mouth or combinations thereof.

In some embodiments, the pre-treatment alkalinity status at the one or more addition sites and/or monitoring sites in the target inland waterbody is low alkalinity, transition alkalinity or high alkalinity. In some embodiments, the pre-treatment alkalinity status at the one or more addition sites and/or monitoring sites in the target inland waterbody is low alkalinity. In some embodiments, the pre-treatment alkalinity status at the one or more addition and/or monitoring sites is transition alkalinity. In some embodiments, the pre-treatment alkalinity status at the one or more addition sites and/or monitoring sites in the target inland waterbody is high alkalinity.

In some embodiments, the one or more addition sites and/or monitoring sites in the target inland waterbody have low pH. In some embodiments, the one or more addition sites and/or monitoring sites in the target inland waterbody have high pH. In some embodiments, the one or more addition sites and/or monitoring sites in the target inland waterbody have low concentration of strong acids. In some embodiments, the one or more addition sites in the target inland waterbody have high concentration of strong acids.

In some embodiments, the pre-treatment alkalinity status (Step C in FIG. 1A) at the one or more addition sites and/or monitoring sites in the target inland waterbody is established after the step of selecting one or more alkaline materials (Step B in FIG. 1A). In some embodiments, the pre-treatment alkalinity status (Step C in FIG. 1A) at the one or more addition sites and/or monitoring sites in the target inland waterbody is established prior to the step of selecting one or more alkaline materials (Step B in FIG. 1A).

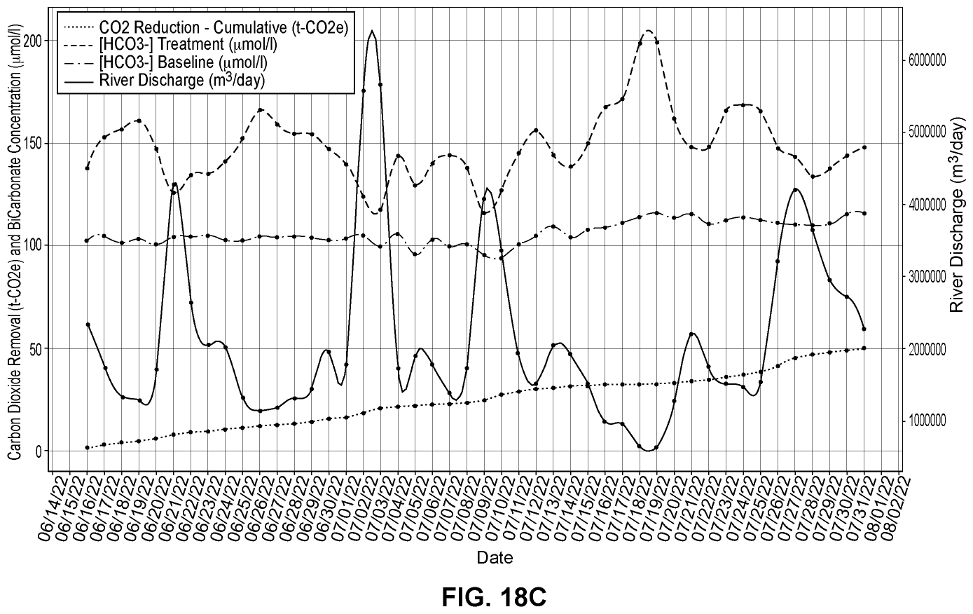

In some embodiments, the one or more methods of adding one or more alkaline materials are determined based on a carbon capture strategy selected from increased carbonation weathering, increased alkalinity, or a combination thereof (FIG. 19). In some embodiments, the carbon capture strategy is increased carbonation weathering. In some embodiments, the carbon capture strategy is increased alkalinity. In some embodiments, the carbon capture strategy is a combination of increased carbonation weathering and increased alkalinity. In some embodiments, the addition of the one or more alkaline materials simultaneously increases alkalinity and the carbonation weathering process in the target inland waterbody, with both processes resulting in a reduction of CO₂ evasion, removal or drawdown of CO₂ from the atmosphere. In some embodiments, the addition of the one or more alkaline materials simultaneously increases alkalinity and the carbonation weathering process in the target inland waterbody, with both processes resulting in a reduction of CO₂ evasion, removal or drawdown of CO₂ from the atmosphere and increased delivery of HCO₃ to the oceans.

In some embodiments, the increased carbonation weathering increases the presence of carbonation weathering reactions to produce a net increase of carbonate anions (HCO₃⁻ or CO₃²⁻) in the target inland waterbody and its drainage waters to the ocean. In some embodiments, the carbonation weathering reaction is shown in Equation (1):

CaMg(CO₃)₂+H₂O+CO₂->Ca²⁺+Mg²⁺+2HCO₃⁻ (1)

In some embodiments, the increased alkalinity increases the efficiency of the delivery of HCO₃⁻ or CO₃²⁻ to the oceans, by increasing the proportion of DIC in the target inland waterbody as HCO₃ and CO₃²⁻ and thus reducing the proportion of H₂CO₃*. In some embodiments, the HCO₃⁻ or CO₃²⁻ is delivered to the ocean for long term storage. In some embodiments, increased alkalinity increases the concentration of DIC in the target inland waterbody. In some embodiments, increased alkalinity increases the concentration of HCO₃⁻ in the target inland waterbody. In some embodiments, increasing the pH in the target inland waterbody above about 6.0 or above about 6.3 drives carbonate speciation away from dissolved CO₂ to anionic species such as bicarbonate (HCO₃⁻) or CO₃²⁻. In some embodiments, increased alkalinity reduces the partial pressure of CO₂ in the water, reduces CO₂ evasion, and therefore increases the target inland waterbody's capacity to uptake CO₂, to store carbon as HCO₃ or CO₃²⁻ (i.e., increasing the target inland waterbody carbonate content). In some embodiments, increased alkalinity increases CO₂ removal/drawdown from the atmosphere, and increases delivery of bicarbonate for long-term storage in the ocean.

In some embodiments, the carbon capture strategy is determined based on a connectivity type of the target inland waterbody to the ocean selected from connected, disconnected or partially connected to the ocean. In some embodiments, the target inland waterbody is connected to the ocean. In some embodiments, the target inland waterbody is partially connected to the ocean. In some embodiments, the target inland waterbody is disconnected from the ocean (FIG. 1C).

In some embodiments, when the connectivity type is connected, the carbon capture strategy is increased carbonation weathering.

In some embodiments, when the connectivity type is disconnected, the carbon capture strategy is increased alkalinity. In some embodiments, when the connectivity type is disconnected, the carbon capture strategy is a combination of increased alkalinity and increased carbonation weathering.

In some embodiments, when the connectivity type is partially connected, the carbon capture strategy is increased carbonation weathering. In some embodiments, when the connectivity type is partially connected, the carbon capture strategy is increased alkalinity. In some embodiments, when the connectivity type is partially connected, the carbon capture strategy is a combination of increased carbonation weathering and increased alkalinity.

In some embodiments, when the target inland waterbody is disconnected or partially connected to the ocean and there is an existing or planned enhanced weathering project in the watershed, the carbon capture strategy is increased alkalinity. In this embodiment, the increased alkalinity strategy in combination with the existing or planned enhanced weathering project in the watershed increases the efficiency of carbonate delivery to the ocean and thereby achieving long-term sequestration of a land-based enhanced weathering project.

In some embodiments, the carbon capture strategy comprises additional CDR pathways such as increasing the alkalinity of the receiving ocean, net ecosystem production, for example where acidification has caused lasting environmental harm, changing rates of dissolved organic carbon mineralization, or a combination thereof.

In some embodiments, the connectivity type of the target inland waterbody to the ocean is determined. In some embodiments, the connectivity is determined based on the established pre-treatment alkalinity status of the target inland waterbody. In some embodiments, the pre-treatment alkalinity status is determined based on at least one of alkalinity, carbon variable, strong acids and pH values of the inland waterbodies that form the drainage network from the one or more addition sites to the river mouth or combinations thereof (FIGS. 1C and 1E).

The alkalinity is a measure of the ability of the water body to neutralize acids and bases and thus maintain a stable pH level. In some embodiments, the alkalinity (Alk) is defined as in Equation (2):

Alk=(HCO₃⁻)+2(CO₃⁻)+(OH⁻)−(H⁺) (2)

wherein molar concentrations are used and alkalinity has units of eq/L. In some embodiments, adding bases adds OH⁻ to the solution, making alkalinity increase. In some embodiments, reducing rates of acid addition, and thus reducing H⁺ addition rates to solution, making alkalinity increase.

In some embodiments, the alkalinity status is classified as:
low (proportion of DIC as H₂CO₃*>90%);
transition (proportion of 90%>H₂CO₃*>10%); and
high (proportion of DIC as H₂CO₃*<10%).

In some embodiments, the alkalinity status is classified as:
low (proportion of DIC as H₂CO₃*>50%);
transition (proportion of 50%>H₂CO₃*>5%); and
high (proportion of DIC as H₂CO₃*<5%).

In some embodiments, the alkalinity status is classified as:
low (proportion of DIC as H₂CO₃*>30%);
transition (proportion of DIC as H₂CO₃* being less than 30% but greater than >10%); and
high (proportion of DIC as H₂CO₃*<10%).

The proportion of DIC as H₂CO₃* is dependent in part on the temperature and salinity of its aqueous environment. In some embodiments, the proportion H₂CO₃* is derived from equations set forth in Chapter 9 of Mook et al. 2000, the entire contents of which are hereby incorporated herein by reference.

In some embodiments, the one or more methods of adding alkaline material comprising adding the alkaline material directly into a waterbody.

In some embodiments, the one or more methods of adding the one or more alkaline materials at the one or more addition sites (Step E in FIG. 1A) comprise using alkaline amendment application infrastructure (for example as in FIG. 1D). The alkaline amendment application infrastructure which can be used in the method of the present disclosure includes any type of infrastructure for delivering the alkaline amendment (the one or more alkaline materials) into the target inland waterbody. Examples of infrastructure include but are not limited to a doser, a reactor, a barge, a boat, a helicopter, a truck, a conveyor belt, an airplane and a drone (FIG. 1D). In some embodiments, the alkaline amendment application infrastructure is selected from a doser, a reactor, a barge and a boat.

In some embodiments, the alkaline amendment application infrastructure is the doser. In some embodiments, the doser automatically or semi-automatically delivers the one or more alkaline materials directly into a waterbody. In some embodiments, the doser delivers the one or more alkaline materials on a continuous, frequent or periodic cycle. In some embodiments, the doser delivers the one or more alkaline materials to the target inland waterbody as a dry powder or as a slurry (the alkaline material is mixed with water). In some embodiments, the doser delivers the one or more alkaline materials to the target inland waterbody having the mean pH of above 6.0. In some embodiments, the doser delivers the one or more alkaline materials to the target inland waterbody having the mean pH of above 7.0. In some embodiments, the doser delivers the one or more alkaline materials to the target inland waterbody having the mean pH of above 6.3.

In some embodiments, the doser is located such that turbulent waters are immediately downstream of the addition site to increase rates of weathering reactions and maximize dissolution.

In some embodiments, the dosing rate of the one or more alkaline materials is between about 0.14 to about 16 mg per L of river water.

In some embodiments, the one or more alkaline materials are added using the doser together with adding the one or more alkaline materials directly to the one or more addition sites in the same target inland waterbody watershed.

In some embodiments, the alkaline amendment application infrastructure is the reactor. In some embodiments, the alkaline amendment application infrastructure is an open reactor. In some embodiments, the alkaline amendment application infrastructure is a closed reactor. The reactor which can be used in the method of the present disclosure includes any type of enclosed or open volume or structure in which a chemical reaction takes place. Examples of reactors include, but are not limited to, structures for separate storage of reactant(s), structures for delivery of gaseous reactants (e.g., flue gas), structures for delivery of aqueous products or reaction medium into the reactor (e.g., water from river), outflow conduit(s) that disperse materials from the reactor into environments, including land surfaces and waterbodies. In some embodiments, the reactor is a single-stage reactor (one volume where chemical reactions take place), a two-stage reactor (two volumes where chemical reactions take place in two stages), a multiple-stage reactor (multiple volumes where chemical reactions take place in multiple), or any combination thereof.

In some embodiments, the reactor can be used when the pH of the target inland waterbody is below about 4.5, when the pH of the target waterbody is between about 4.5 to 6.3, when the pH of the target inland waterbody is above about 6.3, when strong acids are in the target inland waterbody in sufficient concentration to disrupt the carbonation weathering process, or when the pCO₂ in the target inland waterbody is low. In some embodiments, the reactor can be used when the addition site is too close to the ocean for the complete dissolution of the alkaline material to occur before entry to the ocean or larger waterbody. In some embodiments, the reactor is used when the alkaline material settles in the target inland waterbody before dissolution occurs. In some embodiments, the reactor is used when the turbulence or flow in the target inland waterbody is low. In some embodiments, the reactor can be used when quantification of change in carbon variables is not possible downstream due to high levels of dilution (large target inland waterbody), or high background levels of the carbon variables exist in the receiving waters such that a change due to the addition is difficult to detect. In some embodiments, the reactor is used when there is an aesthetic concern of suspended sediment downstream of the addition site.

In some embodiments, the reactor is a two-staged reactor. In some embodiments, the two-staged reactor can be used when the one or more alkaline materials have chemical constituents, metals or impurities that require removal before being released to the target inland waterbody, when strong acids are present in the target inland waterbody in a concentration that would reduce carbonation weathering efficiency below an acceptable level, or any combination of the above.

In some embodiments, the reactor includes a flue gas link to add concentrated CO₂ to a reaction that takes place in the reactor.

In some embodiments, the reactor comprises one or more agitators to speed the chemical reaction in the reactor using any agitators known in the art.

In some embodiments, the alkaline amendment application infrastructure is a barge or a boat. In some embodiments, any other floating structure which temporarily stores one or more alkaline materials can be used for the one or more methods of adding the one or more alkaline materials. In some embodiments, the barge or the boat disperses the one or more alkaline materials directly into the target inland waterbody. In some embodiments, the one or more alkaline materials are dispersed from the barge or the boat into high volume waters, slowly moving waters with a shallow gradient such as in a lake, a headwater lake, or a river, or any combination thereof. In some embodiments, the barge or boat disperses the one or more alkaline materials in the target inland waterbody as a dry powder or as a slurry (the alkaline material is mixed with water).

In some embodiments, the addition of the one or more alkaline materials to the target inland waterbodies includes, but is not limited to, spreading the one or more alkaline materials upon ice, direct addition to a flowing waterbody or its sediments, the use of rotating drums for instream agitation and mixing the one or more alkaline materials, or engineered side channels that redirect water and induce prolonged residency and dissolution.

In some embodiments, the alkaline amendment application infrastructure comprises a range of storage methods, storage capacities, mixing techniques, timing, frequency and rates of delivery.

In some embodiments, carbonation weathering reactions occur in the target inland waterbody. In some embodiments, carbonation weathering reactions occur in the reactor. In some embodiments, carbonation weathering reactions occur in the target inland waterbody and in the reactor (FIG. 1D). In some embodiments, when the carbonation weathering reactions occur in the target inland waterbody, the majority of the reactions occur in the target inland waterbody. In some embodiments, when the carbonation weathering reactions occur in the reactor, the majority of the reactions occur in the reactor.

In some embodiments, the carbonation weathering reactions occur in the target inland waterbody when the barge, the boat, or the doser is used to directly add the one or more alkaline materials to the inland waterbody or when the residence time of water in contact with the one or more alkaline materials is short before being discharged to the receiving inland water body. In some embodiments, the residence time of water in contact with the alkaline material is more than about 20 minutes, less than about 20 minutes, less than about 15 minutes, or less than about 10 minutes.

In some embodiments, the carbonation weathering reactions occur in the reactor when the pH of the target inland waterbody is below about 4.5, the pH of the target inland waterbody is above about 6.3 or above about 6.5, when strong acids are present in the target inland waterbody in concentrations high enough to disrupt carbonation weathering, when pCO₂ concentration of the target inland waterbody is low, when the carbonation weathering reactions are slow, when the alkaline amendment includes sulphides, when the addition site is too close to the ocean for complete dissolution to occur, when the alkaline material settles in the target inland waterbody before dissolution occurs, when the turbulence or flow of the target inland waterbody is slow, when a precise carbonation weathering efficiency rate is required, when there is an aesthetic or ecological concern with increased suspended sediment in the weathering zone downstream of the addition site, or any combination thereof.

In some embodiments, the alkaline amendment addition infrastructure is configured to have a short mean residence time for the one or more alkaline materials when it is in contact with the other reactants and water in the reactor before it is released to the waterbody, such that most of the weathering reactions take place in the inland waterbody. In some embodiments, the alkaline amendment addition infrastructure is configured to have a longer mean residence time for the one or more alkaline materials when it is in contact with the other reactants in the reactor, such that most of the weathering reactions take place in the reactor, or in both the reactor and in the inland waterbody. In some embodiments, reactors have a longer mean residence time relative to dosers, barges, boats and other forms of direct application.

In some embodiments, reactors are used to increase the speed and efficiency of the carbonation weathering reactions through manipulation of at least one of the pCO₂, pH, pressure, temperature, ionic strength, concentration of strong acids, or by using more reactive alkaline materials in isolation or combination with less reactive alkaline materials.

In some embodiments, concentrated CO₂ is added to the reactor. In some embodiments, the source of the concentrated CO₂ for the carbonation weathering reaction is from an external source (flue gas, or gas emanating from breakdown of organic matter, or from groundwater).

In some embodiments, the source of the CO₂ in the carbonation weathering reaction is from H₂CO₃* contained in the target inland waterbody or from CO₂ contained in the atmosphere.

In some embodiments, the step of adding the one or more alkaline materials further includes establishing bicarbonate concentration at a point of interest and/or near the river mouth of the target inland waterbody watershed (FIG. 1B). In some embodiments, the bicarbonate concentration is established through direct measurement of one or more carbon variables, through modelling of the bicarbonate concentration, or through direct measurement of bicarbonate via a sensor.

In some embodiments, the one or more alkaline materials are added to the one or more addition sites at a frequency ranging from continuous frequency (e.g., every 1 minute or less) to bi-annual (approximately every two years). In some embodiments, the continuous frequency is every 3 minutes, every 5 minutes, every 10 minutes, values in between and the like. In some embodiments, the frequency of adding the one or more alkaline materials is monthly, every 3 months, once a year values in between and the like. It is to be understood that the frequency of the addition of the one or more alkaline materials is within the consideration of the person skilled in the art and any frequency is encompassed within the scope of the present disclosure.

In some embodiments, the one or more alkaline materials are added to the target inland waterbody as a dry powder. In some embodiments, the one or more alkaline materials are added to the target inland waterbody as a slurry (the alkaline material is mixed with water).

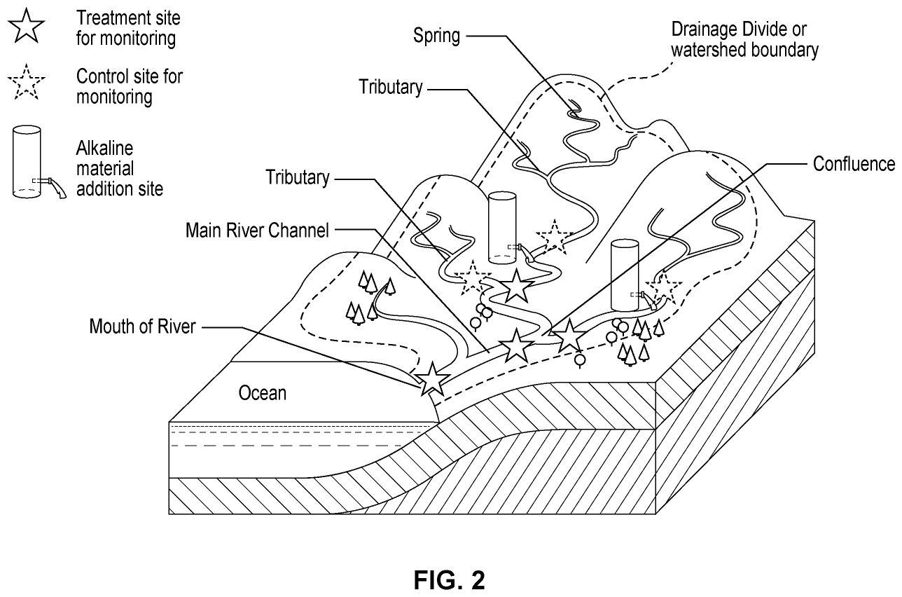

In some embodiments, the step of adding the one or more alkaline materials further includes monitoring at one or more monitoring sites (e.g., FIG. 1E and FIG. 2). In some embodiments, the one or more monitoring sites include one or more treatment sites and one or more control sites, the latter of which are sites not affected by the alkaline treatment that are used to infer the background condition of the treatment sites (FIG. 2). In some embodiments, the background conditions are conditions that are projected to occur at treatment sites had the treatment not taken place. Examples of monitoring site and background conditions include values of water variables, carbon variables or ecological variables.

In some embodiments, the one or more monitoring sites include one treatment site, two treatment sites, three treatment sites or more. In some embodiments, the one or more monitoring sites include one control site, two control sites, three control sites or more.

In some embodiments, the one or more treatment sites are located downstream of the one or more addition sites. The treatment sites can be located for example, below the weathering zone, at the river mouth, at the outflow of the one or more addition sites and between the one or more addition sites.

In some embodiments, the one or more control sites are located upstream of the one or more addition sites.

In some embodiments, the method of the present application further includes testing one or more of carbon variable, ecological variable and water variable values at the one or more monitoring sites to determine the response of the target inland waterbody and/or its inland waterbodies that connect the one or more addition sites of the one or more alkaline materials to the river mouth.

In some embodiments, the method of the present application further includes establishing target ranges of one or more of the carbon variable, ecological variable and water variable values at the one or more monitoring sites. The target range is a range of values of one or more of the water variables, ecological variables and/or carbon variables that are chosen to reduce at least one of the risk of environmental harm, increase environmental co-benefits, optimize CDR, optimize cost-benefit outcomes, or any combination thereof.

In some embodiments, the method of the present application further includes monitoring or estimating the one or more of the carbon variable, ecological variable and water variable values at the one or more monitoring sites.

In some embodiments, the method of the present application further includes calculating baseline values of the one or more of the carbon variable, ecological variable and water variable values at the one or more monitoring sites.

In some embodiments, the one or more of the CDR, carbon variable, ecological variable and water variable values at the one or more monitoring sites are compared with the target ranges of said one or more of the carbon variable, ecological variable and water variable values. In some embodiments, when the one or more of the carbon variable, ecological variable and water variable values at the one or more monitoring sites are not within the target value of said one or more of the carbon variable, ecological variable and water variable values, the one or more alkaline material addition methods are adjusted to achieve values within the target range. In some embodiments, the method of the present application further includes monitoring or estimating CDR, pCO₂, pH, water temperature, salinity, discharge and HCO₃⁻.

In some embodiments, when CDR value is not within the target value, the alkaline material addition method is adjusted to achieve CDR values within the target range. Adjustments may include having the weathering take place in a reactor where pCO₂ levels are elevated, using microbial catalysts for carbonation weathering, increasing the dose of alkaline amendment, and changing the type of alkaline amendment.

In some embodiments, the method of adding the one or more alkaline materials is adjusted by adding a non-carbonate based alkaline amendment, neutralizing strong acids, or increasing the alkalinity in the drainage waters leading from the addition sites to the river mouth.

In some embodiments, in cases where carbonate minerals comprise the alkaline amendment, because of the presence of strong acids and/or low pH the alkaline amendment strategy must be adjusted so that strong acid weathering reactions do not cause a release CO₂ from carbonate minerals. As an example, CO₂ is produced as in Equation (3), where the reactant is calcite (CaCO₃) and the acid anion is from a strong acid (HNO₃⁻).

CaCO₃+2HNO₃⁻<->Ca²⁺+2NO₃⁻+H₂O+CO₂ (3)

In some embodiments, the carbon variable monitored or indirectly estimated at the one or more monitoring sites is bicarbonate concentration.

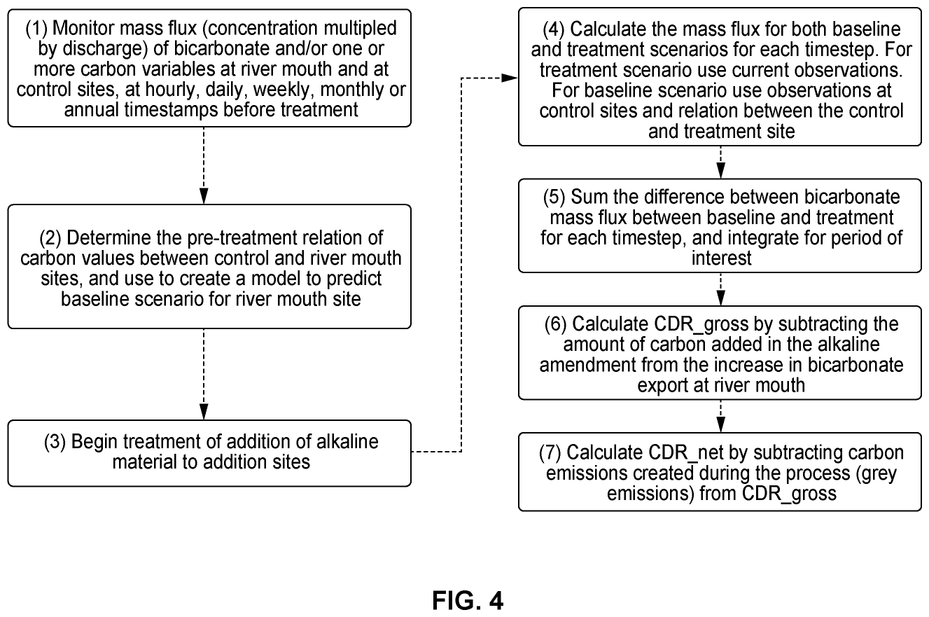

In some embodiments, the CDR values are calculated as in FIG. 4, and as follows:

CDR_net=C−I−G (4)

where
CDR_net is the carbon dioxide removed by the project (in t CO₂ₑ/Δt)
C is increase in HCO₃⁻ delivery to ocean (in t CO₂ₑ/Δt)
I is amount of carbon added in alkaline amendment (in t CO₂ₑ/Δt)
G is carbon expended in grey emissions (in t CO₂ₑ/Δt)
Δt is period of interest, can range from minutes to months to years
CO₂ₑ is the equivalent mass of carbon dioxide
   and where

C=(HCO₃⁻ₜ−HCO₃⁻_{b})(Q) (5)

where
C is the increase in HCO₃ delivery to the ocean at the river mouth (in t CO₂ₑ/dt)
HCO₃⁻ₜ is concentration at river mouth during treatment (in t CO₂ₑ/volume)
HCO₃⁻_{b} is concentration at river mouth for baseline scenario (in t CO₂ₑ/volume)
Q is river discharge (in volume/dt)
dt is timestep of calculation, typically ranging from minutes to weeks
CO₂ₑ is the equivalent mass of carbon dioxide

The term “grey emissions” as used herein refers to all CO₂ emissions that occur from the project and which must be removed from the estimated CDR total. Grey emissions include those emissions described by the US EPA GHG Inventory Development Process and Guidance document (https://www.epa.gov/climateleadership/ghg-inventory-development-process-and-guidance). Scope 1 emissions are direct greenhouse (GHG) emissions that occur from sources controlled by the project organization (e.g., fuel combustion onsite, maintenance vehicles, etc.). Scope 2 emissions are indirect GHG emissions associated with the purchase of energy such as electricity that is required to operate a project. Scope 2 emissions physically occur at the facility where they are generated but they are accounted for in an organization's GHG inventory because they are a result of the organization's energy use. Scope 3 emissions are the result of activities from assets not owned or controlled by the project organization and not covered by scope 1 or scope 2, sometimes referred to as value chain emissions, examples include fuel, waste and energy needs required to manufacture and deliver components needed to operate the project.

In some embodiments, the target value for CDR is such that is greater than 0. In some embodiments the target value is to have C greater than (D+G), following the definitions above.

In some embodiments, the one or more alkaline materials are added to the one or more addition sites prior to developing carbon capture strategy and/or addition method. In some embodiments, the one or more alkaline materials are added to the one or more addition sites after developing the carbon capture strategy and/or the addition method.

In some embodiments, the amount of the one or more alkaline materials is effective to enable enhanced weathering to occur and to increase the pH of the target watershed downstream of the one or more addition sites above about 5.0 is determined by the dosage and frequency of addition of the one or more alkaline materials. In some embodiments, the dosage [g_aa in Equation (6)] is about 0.1 mg/L to about 100 mg/L, less than about 1 mg/L, higher than about 100 mg/L, about 3 mg/L to about 50 mg/L, about 5 mg/L to about 30 mg/L, or about 7 mg/L to about 20 mg/L, suitably about 3 mg/L to about 15 mg/L, and is added based on site-specific data including calculated constants for solubility, discrete estimates of ambient alkalinity and real-time measurements of discharge, carbon variables, and stream pH. In some embodiments, the dosage and frequency are determined via a real-time iterative dosing method which facilitates real-time optimization and also real-time auditing of CO₂ drawdown/removal to reduce the reliance on estimates of CO₂ drawdown/removal via algorithms and remote sensing.

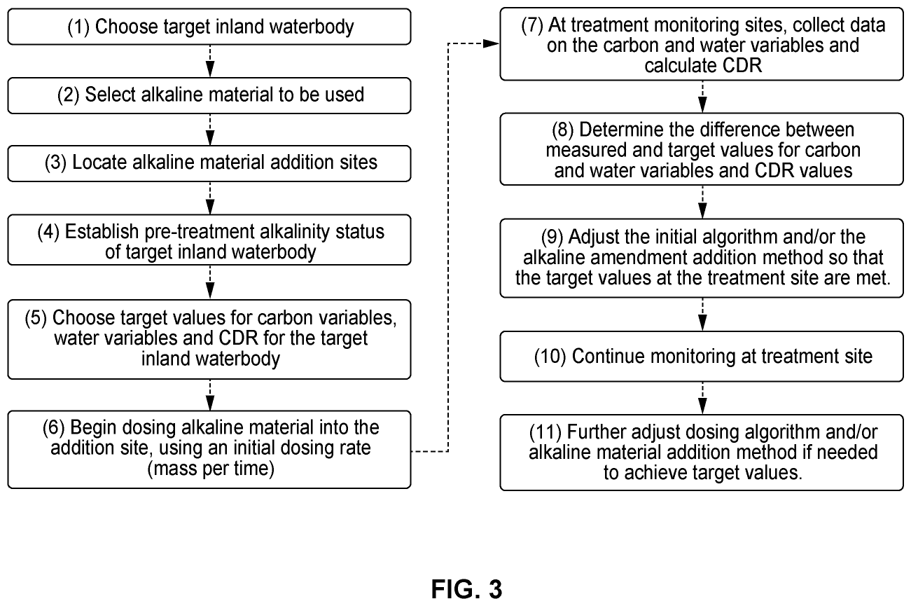

In some embodiments, the real-time iterative method comprises creation of dose/response curves for each target site for the dose and discharge and the one or more response variables. In some embodiments, the one or more response variables are selected from, but are not limited to, pH, and carbon variables. Therefore, in some embodiments, if the downstream sensor detects that one or more of the response variables are not in a target range, it would signal to a doser to increase or decrease the dose (as in FIG. 3). In some embodiments, the increase/decrease is calculated as a function of the deviation of the level from the target level, the dose/response curve, and the discharge at the time.

In some embodiments, the one or more alkaline materials are added to the one or more addition sites using a dosing algorithm. The dosing algorithm includes specific calculations and/or formulae that are used to automate the release of the one or more alkaline materials into the target inland waterbody.

A dosing algorithm is used to control the rate of alkaline addition based on one or all of continuous monitoring, modelling, periodic measurements, and a starting estimate of the concentration of alkaline product needed to achieve the desired downriver pH change, following FIG. 3 and Equation (6):

D=Q*g_aa (6)

where
D is dosage of alkaline amendment in mass per time, e.g., kg*s⁻¹
Q is river discharge at the addition point, in volume per time, e.g., m³*s⁻¹, obtained from field measurements or forecast from a model, or as estimated by river stage
g_aa is concentration of alkaline amendment, in mass per volume of water (e.g., in kg*m⁻³), or mass per river stage

The initial value of g_aa is based on laboratory dose-response curve or an assumed concentration (e.g., 1 mg·L⁻¹). g_aa is then adjusted after alkaline material has been added, based on values of carbon and water variables at treatment sites, including bicarbonate amounts, and/or calculated CDR values. g_aa will vary with type of alkaline material. Since g_aa is a function of both discharge and chemical properties which vary through time, chemical properties need to be monitored regularly in order to make adjustments to g_aa to achieve target levels of CDR and other carbon variables. Monitoring and adjustments can occur at a high frequency (e.g., every minute) or low frequency basis (e.g., every year), depending on the nature of the river, for example, whether the river flow and chemistry have a high coefficient of variation on a daily/monthly basis.

In some embodiments, the dosing algorithm variable Q is developed based on at least one of the water inputs to the target inland waterbody via precipitation, anthropogenic water inputs, water released from an impoundment, or snowmelt, pH, discharge or stage of the target inland waterbody. In some embodiments, the variable g_aa is based on the value of one or more carbon variables, for example, bicarbonate concentrations, or the value of one or more water variables or ecological variables. In some embodiments, the values of the variables used for the dosing algorithm are received from direct measurements, from statistical relations or models, or from process models. In some embodiments, the models may include rainfall-runoff models, hydrological models, biogeochemical models, reactive transport models, carbon models, ecological models, climate models, or any combination thereof.

In some embodiments, the values used as inputs to the dosing algorithm are obtained from one monitoring site, multiple monitoring sites, or values from continuous measuring along a drainage network extending from the addition point to the river mouth. In some embodiments, when the river mouth contains the addition site of the target inland waterbody, the river mouth comprising the addition site of the target inland waterbody.

In some embodiments, the dosing algorithm is a linear relation. In some embodiments, the dosing algorithm is a non-linear relation, for example, where g_aa is a function of Q. In some embodiments, the dosing algorithm is a stochastic relation. In some embodiments, the dosing algorithm is derived via machine-learning, deep-learning, or AI.

In some embodiments, the one or more methods of adding the one or more alkaline materials for the one or more addition sites is determined by establishing an initial alkaline material addition method for the one or more addition sites, testing the one or more treatment sites to determine if water and/or carbon variables are the within target values after one or more alkaline materials have been added and adjusting the one or more alkaline material addition methods and/or one or more alkaline materials, so that the carbon and/or water variables are within target range of said values at the one or more treatment sites. In some embodiments, one or more methods of adding the one or more alkaline materials is determined for each one of the one or more addition sites.

In some embodiments, the dosage and the frequency of application of the one or more alkaline materials addition is determined based on the volume of water being treated or discharge rates, flushing rate, temperature and stratification, water chemistry, carbon variable values, water variable values and/or particle size of the one or more alkaline materials.

In some embodiments, the dosage and frequency of addition of the one or more alkaline materials is further determined based on at least one of carbon variables, water variables, such as for example, temperature, dissolved organic carbon (DOC), HCO₃⁻ concentration or CO₂ evasion rates of the water downstream of the one or more addition sites.

In some embodiments, the initial alkaline material type is selected by testing the one or more water variables and/or carbon variables at the one or more monitoring sites and by comparing values of select water and/or carbon variables to their target ranges. In some embodiments, the initial alkaline material type is selected by using a model.

In some embodiments, the method of the present application further includes calculating carbon capture by estimating the amount of CO₂ drawdown/removal from the addition of the one or more alkaline materials to the target inland waterbody. In some embodiments, the carbon capture is calculated by estimating the amount of CO₂ drawdown/removal from carbonation weathering processes in the target inland waterbody [Equation (7)]. In some embodiments, the carbon capture is calculated by estimating the amount of CO₂ drawdown/removal from carbonation weathering processes and change in alkalinity in the target inland waterbody. The CO₂ drawdown/removal is the amount of CO₂ equivalent in mass per time that has been removed from the atmosphere and delivered to oceans for storage.

CDR_net=(F*E*D)−G (7)

where
CDR_net is the net amount of carbon removed during the project (in t CO₂ₑ/Δt)
F is CO₂ drawdown/removal during carbonation weathering of stoichiometric ratio of carbonate, calculated as the mass of CO₂ drawdown/removal per mass of alkaline amendment that underwent carbonation weathering (e.g., 0.48 for dolomite) (dimensionless)
D is the mass of alkaline amendment added (in t CO₂ₑ/Δt)
E is carbonation weathering efficiency (1 or less) (dimensionless)
G is carbon expended in grey emissions (in t CO₂ₑ/Δt)
Δt is period of interest
CO₂ₑ is the equivalent mass of carbon dioxide

In some embodiments, when carbon capture is calculated by estimating the amount of CO₂ drawdown/removal from carbonation weathering processes, the mass of the one or more alkaline materials is added to the inland waterbody per unit of time, multiplied by the stoichiometry of the carbonation weathering reaction determines the amount of H₂CO₃*per mass of the one or more alkaline materials added [Equation (7)].

In some embodiments, when carbon capture is calculated by estimating the amount of CO₂ drawdown/removal from carbonation weathering processes, when the alkalinity status of some or all of the waterbodies between the addition point and the river mouth have an alkalinity status of “low” or “transition”, the carbon capture calculations include a parameter that represents the proportion of DIC speciated as HCO₃⁻ and CO₃²⁻, as a function of one or more of pH, salinity, temperature, ionic strength and pressure.

In some embodiments, the CO₂ drawdown/removal is estimated by calculating the change in carbon variable concentrations caused by the addition of alkaline amendment multiplied by the discharge at the river mouth [Equations (4) and (5)].

In some embodiments, the CO₂ drawdown/removal at the river mouth is estimated by multiplying the increase in HCO₃⁻ and CO₃²⁻ by the discharge at the river mouth for a period of time.

In some embodiments, the carbon capture calculations estimate CO₂ drawdown/removal for a period from less than one hour to over multiple years.

In some embodiments, the carbon capture calculations are based on observations. In some embodiments, the carbon capture calculations are based on modelling, AI, deep learning, or machine learning.

In some embodiments, estimating CO₂ drawdown includes testing the target inland waterbody, its drainage waters and inland waterbodies in neighboring watersheds at one or more monitoring sites.

In some embodiments, the carbon drawdown accounted for is only that in the carbonation weathering equation or the increased capacity of the inland waters to hold CO₂, but not accounting for both mechanisms.

In some embodiments, the one or more monitoring sites comprising one or more treatment sites, control sites, or any combination thereof (FIG. 2).

In some embodiments, when the inland waterbodies connecting the addition site with the river mouth contain one or more low or transition alkalinity sections, the treatment sites are located at downstream end of a low or transition alkalinity waterbody segment. In some embodiments, the treatment sites are located at the bottom of the weathering zone and at the river mouth. In some embodiments, the treatment monitoring sites are located at the river mouth (FIG. 1E).

In some embodiments, when the waterbody is a transition or high alkalinity waterbody, the treatment sites are located at downstream end of weathering zone (FIG. 1E). In some embodiments, the treatment sites are located at the river mouth (FIG. 1E). In some embodiments, the treatment sites are located as shown in FIG. 1E: a) for conditions where the carbon capture strategy is to have part of all of the carbonation weathering reactions occurring in the target inland waterbody, and for conditions where the alkalinity status is low or transition; b) for conditions where the carbon capture strategy is to have part of all of the carbonation weathering reactions occurring in the target inland waterbody, and for conditions where a low or transition alkalinity waterbody flows into a transition or high alkalinity waterbody; c) for conditions where the carbon capture strategy is to have most of the carbonation weathering reactions occurring in a reactor and for conditions where the alkalinity status of the waterbodies connecting the addition site with the target inland waterbody mouth are transition or high alkalinity, and d) for conditions where the carbon capture strategy is to have most of the carbonation weathering reactions occurring in the target inland waterbody and for where the alkalinity status of the waters connecting the addition site and the target inland waterbody mouth is transition or high.

In some embodiments, the treatment site is located at the outflow of the reactor. Examples of locating the treatment site at the outflow of the reactor include estuaries with high alkalinity, weathering reactions taking place inside a reactor, with high target inland waterbody discharge such that it is difficult to detect a change in concentration of carbon variables, or in other cases where it is difficult to detect a change in concentration of carbon variables.

In some embodiments, the baseline conditions are estimated in the inland waters connecting the addition site with the river mouth. In some embodiments, the baseline conditions are estimated using modelling. In some embodiments, the baseline conditions are estimated using direct observations, or a combination of direct observations and modelling.

In some embodiments, when the weathering reactions occur in the reactor, the baseline conditions are established from the target inland waterbody chemistry directly upstream of the reactor, or inflow to reactor if using water from the target inland waterbody for the reaction.

In some embodiments, when the weathering reactions occur in the target inland waterbody, the control sites and treatment sites are tested to establish pre-treatment values for one or more of the carbon variables, ecological variables and water variables, the pre-treatment data from the control sites and the treatment sites are tested and the models/relationship between treatment and control sites are established to enable future prediction of baseline conditions at treatment sites.

In some embodiments, the change in the carbon variables at the monitoring site(s) is calculated by comparing the difference between the treatment vs baseline values of carbon variables or by multiplying the difference in the carbon variables by the target inland waterbody discharge, for timesteps ranging from one minute to multiple years [as in the example in Equation (5)].

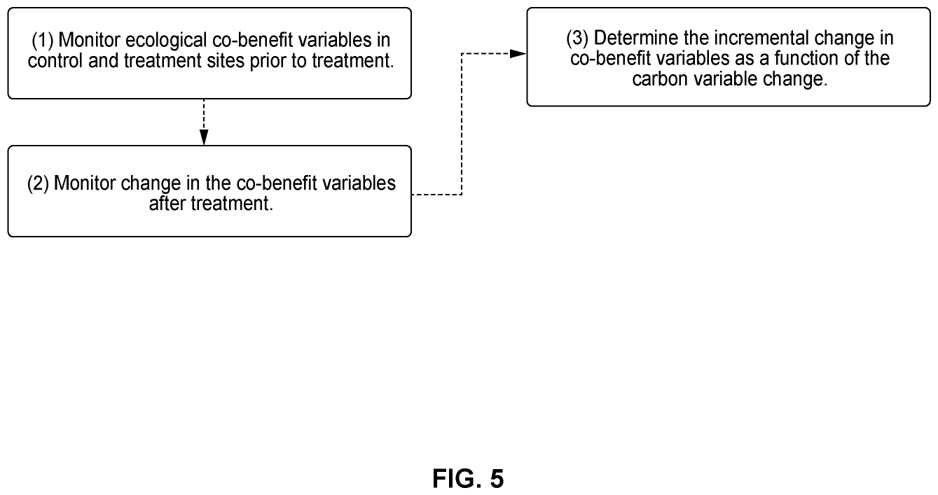

In some embodiments, the ecological variables are measured to quantify additional ecosystem co-benefits (FIG. 5).

The following examples of the present disclosure are intended to be illustrative but non-limiting:

### EXAMPLES

### Methods of Removing Atmospheric Carbon Dioxide (CO₂)

Table 1 below illustrates the steps of the method of removing atmospheric carbon dioxide (CO₂) in two exemplary target inland water bodies, one of low alkalinity (Acidic river) and one of neutral/high alkalinity (Alkaline/neutral river). The details of each step are provided below. The examples are based on a combination of field observations and modelled data.

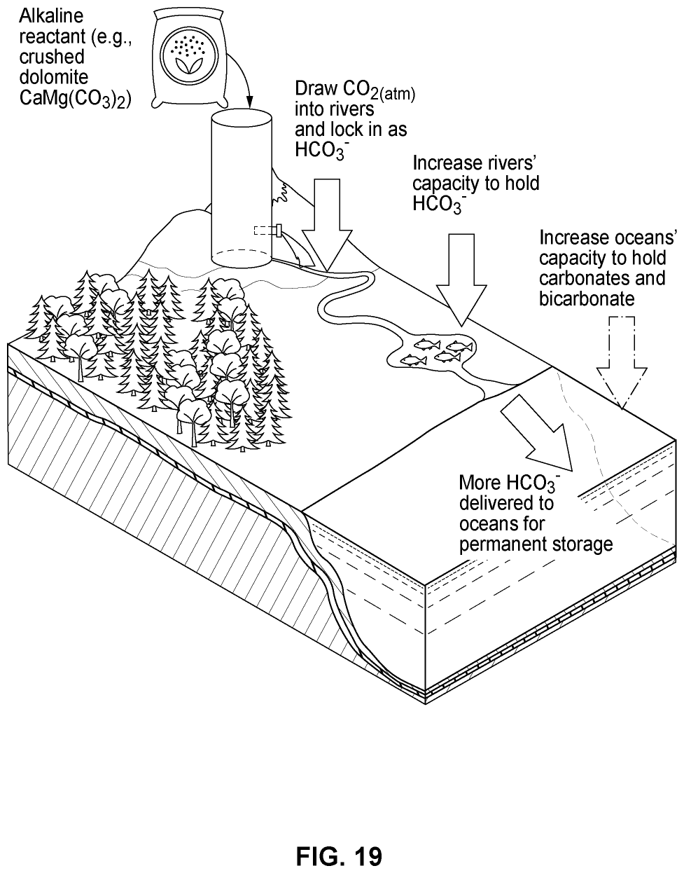

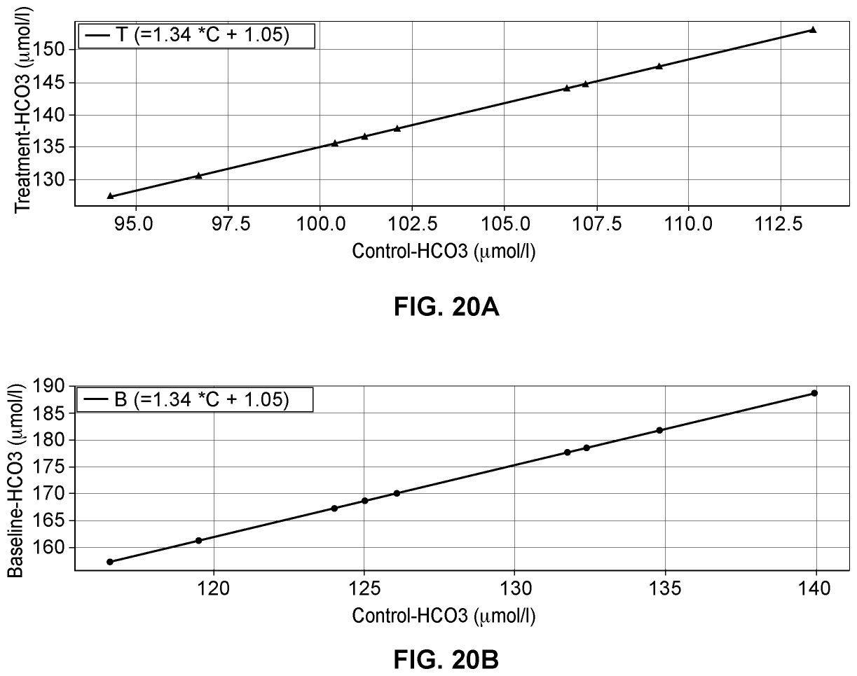

TABLE 1
Illustration of the steps of the of the method of removing atmospheric carbon dioxide
(CO₂) in low alkalinity and neutral/high alkalinity target inland waterbodies.

| | Case A | Case B |
|---|---|---|
| Step (From FIG. 1A) | Acidic river | Alkaline/neutral river |
| A. Identify target inland | West River | North River |
| waterbody | | |
| B. Establish addition site(s) | Two, in headwaters; alkaline | One, in lower reach; alkaline |
| and select alkaline material to | material is crushed dolomite | material is crushed dolomite |
| be used | | |
| C. Establish pre-treatment | Low alkalinity, low pH, | Transition/high alkalinity, |
| alkalinity status. Test target | elevated strong acids: low | neutral/high pH, low strong |
| inland waterbody pre- | alkalinity status, and | acids; high alkalinity status |
| treatment at control and | disconnected connectivity | and connected connectivity |
| treatment sites | type | type |
| D. Establish control sites and | Upstream control | Upstream control, baseline |
| baseline models | | model as in (FIG. 20) |
| E. Add alkaline material to | Doser application of crushed | Direct application of dolomite |
| addition sites | dolomite following Equation 6 | following Equation 6 and |
| | and values in Table 3A | values in Table 3C |
| F. Monitor carbon and water | CDR < 0; target not met | CDR > 0; target met |
| variables, calculate CDR | | |
| G. Adjust application method | Modified by pre-treating river | None needed |
| to achieve targets | water before adding dolomite | |
| H. Recalculate CDR | CDR > 0; target achieved | CDR remains > 0 |
| | with modifications | |

### Step A. Identify Target Inland Waterbody

Both target inland waterbodies described in this example are well-connected to the Atlantic Ocean, are widely accessible along the drainage network, and both have a hydrological discharge rate suitable for the addition of alkalinity. Case B has favorable water chemistry for carbonation weathering reactions, while Case A does not.

### Step B. Test Target Inland Waterbody and Identify One or More Addition Site(s) and Select Alkaline Material to be Used

For Case A, two addition sites were selected. The longitudinal slope of the river drainage network was obtained from a digital elevation model, and reaches with higher slope suitable for addition sites were identified. Lakes were mapped in the drainage network. The road network was identified and addition sites were located so that they were close to the road network. Potential anthropogenic impacts were examined; no dams or point source pollution outflows were identified. The mean annual discharge was measured from neighbouring hydrometric stations and a watershed model. The channel and floodplain boundaries around the addition sites were mapped. The ecology was examined and the watershed was found to have Atlantic salmon populations. The bedrock type was examined and it was found to be underlain by slate and granite bedrock. Wetland distribution was mapped.

For Case B, one addition site was selected to be 10 km above the river mouth. The longitudinal slope of the river drainage network was obtained from a digital elevation model and reaches with higher slope suitable for an addition site was identified. Lakes were mapped in the drainage network. The road network was identified and addition sites were located so that they were close to the road network. Potential anthropogenic impacts were examined; no dams or point source pollution outflows were identified. The mean annual discharge and flood frequency analysis was established from neighbouring hydrometric stations and a watershed model. The channel and floodplain boundaries around the addition sites were mapped. The ecology was examined and the watershed was found to have Atlantic salmon populations. The bedrock type was examined and it was found to be underlain by dolomitic limestone. Wetland distribution was mapped.

### Step C. Establish Pre-Treatment Alkalinity Status of the Target Inland Waterbody at Control and Treatment Sites Including at River Mouth.

Case A was tested and found to have low alkalinity, low pH (mean pH was 4.7) and elevated strong acids (e.g., DOC concentrations of 15 mg/L). Its alkalinity status was found to be “low”, and pre-treatment connectivity status was determined to be “disconnected” (as in FIG. 1C). Case B was tested and found to have high alkalinity, high pH (mean pH of 7.4), and low strong acids (e.g., DOC concentrations of less than 5 mg/L); its alkalinity status was found to be “high” and pre-treatment connectivity status was determined to be “connected” (as in FIG. 1C).

### Step D. Establish Models Between Control and Treatment Sites to Enable Future Prediction of Baseline Values.

Case A was tested and it was determined to set control sites directly upstream of both addition sites, and have three treatment sites, one below the weathering zone, one at the river mouth, and one between the two sites. The connectivity type below the addition site was determined to be disconnected (FIG. 1C, case d, disconnected).

Case B was tested and it was determined to set control sites directly upstream of the addition site and to set the treatment sites at the outflow of the addition site and at the river mouth, considering the connected status of the drainage waters (following FIG. 1C, case c, connected).

Baseline values were calculated as follows: first, a pre-treatment relation was established between a river-mouth treatment site (T) and the control site (C) (e.g., FIG. 20A). In this example for Case B, the relation was a linear model between bicarbonate concentrations at the control and treatment sites where T=1.34C+1.05, where units are μmol/L. Following application of the alkaline amendment, the baseline values were calculated from the control values, using the model established pre-treatment (FIG. 20B).

### Step E. Add Alkaline Material to Addition Sites, Following the Initial Alkaline Material Addition Method

Case A: The initial method for the addition of alkaline materials for Case A was done via automated lime dosing at two locations in the upper watershed, using method for improving fish habitat in acidic rivers, by dosing to achieve target pH instead of target CDR. These dosers were situated such that turbulent waters were immediately downstream of the addition sites in order to increase rates of weathering reactions and maximize dissolution. Dosing rates of crushed dolomitic limestone ranged between 880 and 7900 mg/m³ and accounted for the dissolution of the product (Table 3A). Total dosing amounts are 23 tonnes of dolomite over the study period. Purity of the dolomite was monitored via laboratory analyses. Depending on river gradient and the hydrology of the watershed, the optimal method to distribute alkalinity may be: (a) automated lime dosers situated along a drainage network, (b) the addition of alkaline material directly to headwater lakes, or (c) a combination of the two approaches. The CDR strategy for Case A is to increase both the carbonation weathering rates by adding dolomite and to increase the alkalinity to increase retention of HCO₃⁻ in the river downstream of the addition site to the river mouth.

Case B: Alkaline materials for Case B were added via automated lime dosing, using the same infrastructure as for Case A, at a location in a lower reach of the watershed. Dosing rates of crushed dolomitic limestone ranged between 880 and 7814 mg/m³ and accounted for the dissolution of the product (Table 3A). Total dosing amounts are 230 tonnes of dolomite over the study period. Purity of the dolomite was monitored via laboratory analyses. The CDR strategy for Case B was to increase carbonation weathering rates only as the alkalinity downstream is already high.

### Alkaline Material

The alkaline material used in both Case A and Case B examples was dolomitic limestone CaMg(CO₃)₂ with approximately 40% calcium and 33% magnesium, by weight.

### Step F. Monitor Water and Carbon Variables and Calculate CDR

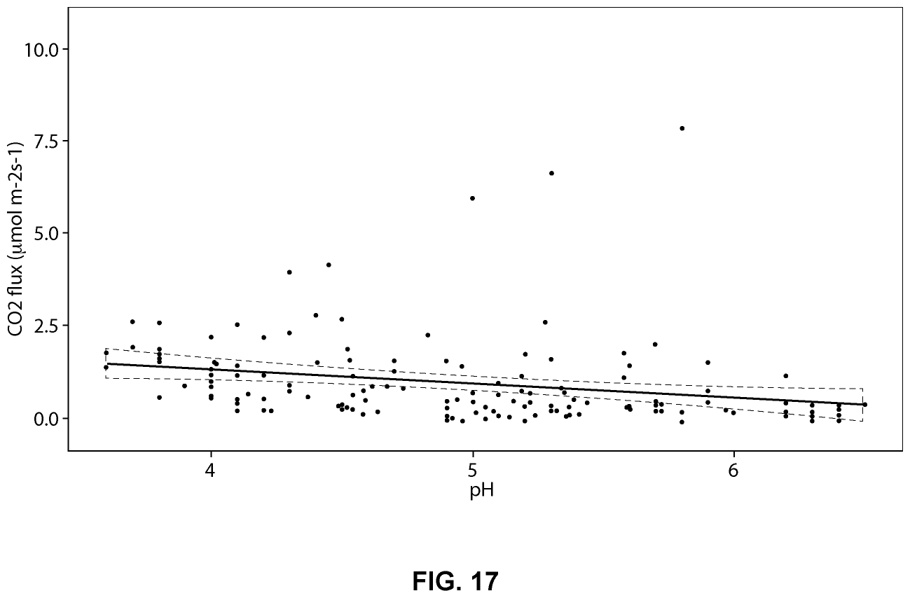

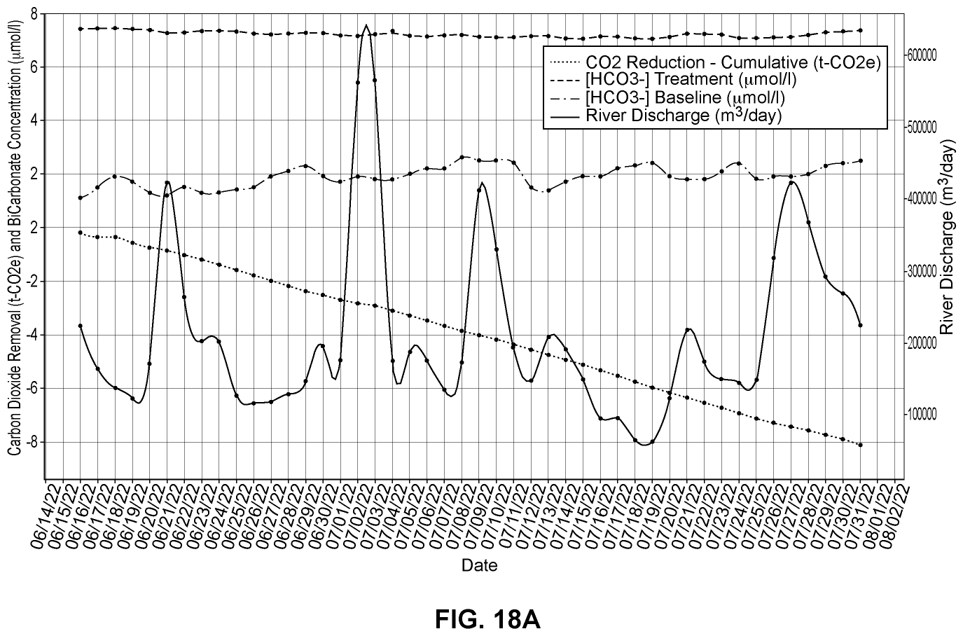

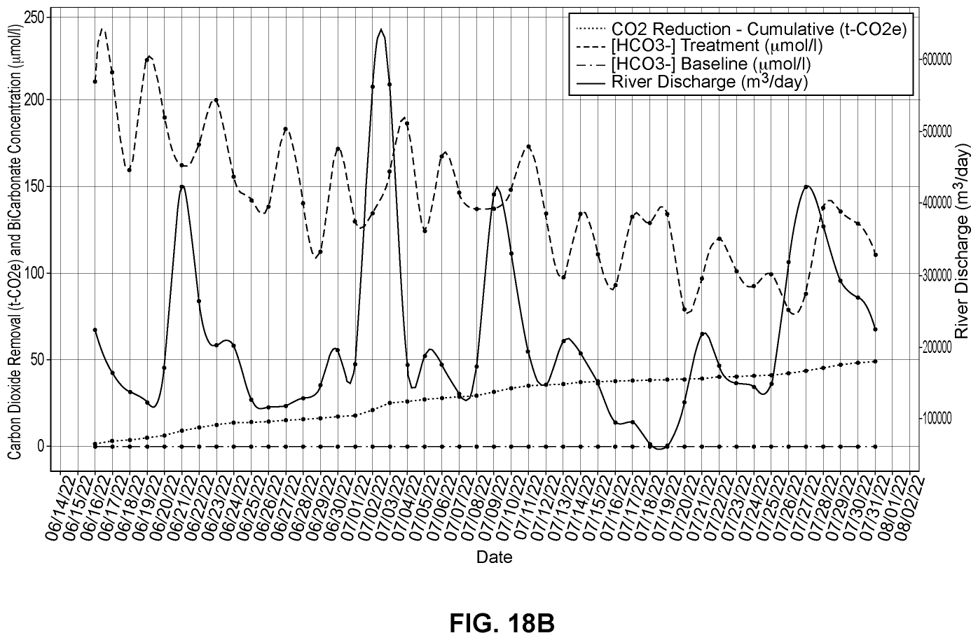

The results for the control and treatment sites for both Case A and B were examined (Tables 2 and 3 and FIG. 18). Baseline values were calculated from control data using the relation between the control and treatment sites established during the pre-treatment phase (FIG. 20). At the control and treatment sites, pCO₂, pH, water temperature, salinity and discharge were measured, and HCO₃ was calculated using the program CO₂SYS (Lewis and Wallace, 1998) (Table 3). Bicarbonate concentration differences between the treatment and baseline values at the river mouth were multiplied by discharge at the river mouth on a daily timestep to quantify the increase in bicarbonate export flux [Equation (5)]. From this increase in bicarbonate export flux the amount of carbon added in the dolomite amendment and the amount of grey emissions were subtracted [Equation (4)]. The CDR totals were then summed for the period of interest (Table 2).

Table 2 provides CDR calculations for the Case A initial alkaline amendment application method (Step F) and improved alkaline amendment application method (Step H), and for Case B, initial and final alkaline amendment application. Cases were modelled for 43-day periods for scenarios based on observed data. All masses are in metric tonnes (t) of CO₂ₑ. d=day. C is the increase in HCO₃ delivery to ocean. I is amount of carbon added in alkaline amendment. G is carbon expended in grey emissions. CDR_gross does not account for grey emissions. CDR_net does account for grey emissions. Totals are the summed masses over the 43-day period. Potential carbon drawdown from increased ocean alkalinity was not considered here (as in FIG. 19).

TABLE 2
CDR calculations.

| | CASE A STEP F | | CASE A STEP H | | CASE B | |
|---|---|---|---|---|---|---|
| | | Mean | | Mean | | Mean |
| | Total | daily | Total | daily | Total | daily |
| | t | t/d | t | t/d | t | t/d |
| C | 2.3 | 0.049 | 59.7 | 1.3 | 156 | 3.39 |
| I | 10.6 | 0.23 | 10.6 | 0.23 | 108 | 2.3 |
| CDR_GROSS | −8.1 | −0.18 | 49.3 | 1.07 | 50.0 | 1.09 |
| G | 1.6 | | 1.6 | | 1.6 | |
| CDR_NET | −9.7 | | 47.7 | | 48.3 | |

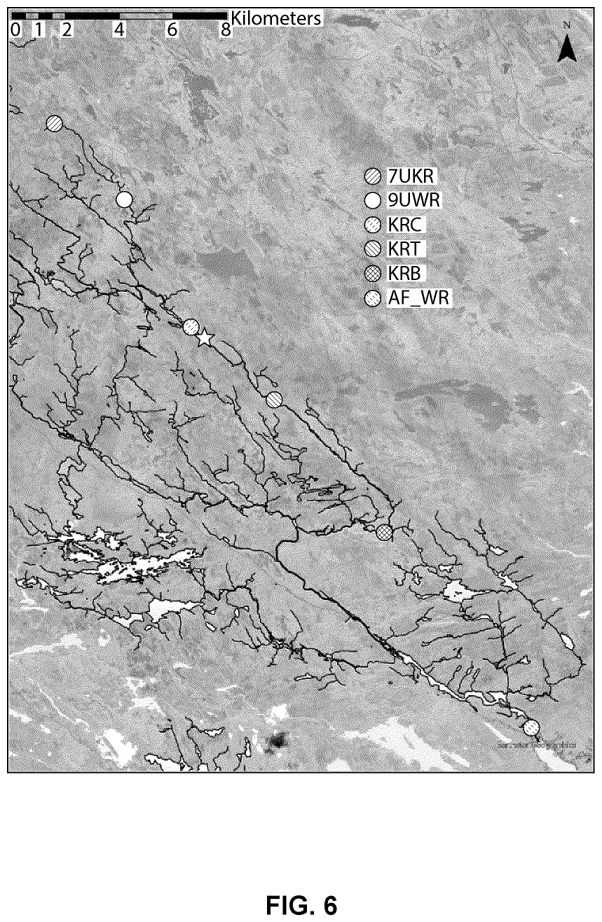

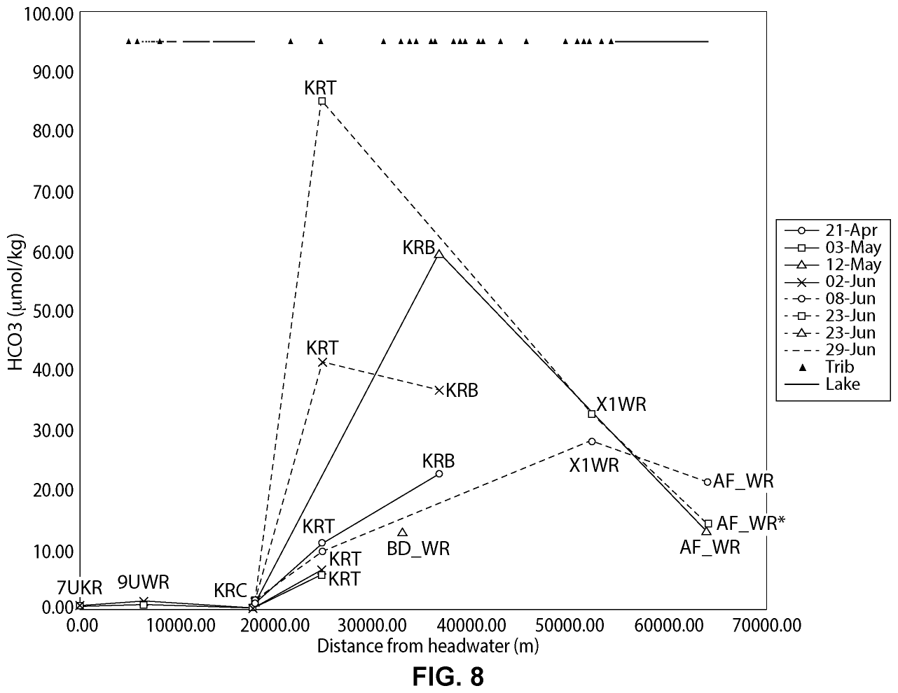

In Case A, for Step F, the CDR was negative (FIG. 18A, Table 2). The CDR value was negative because the increase in CO₂ₑ at the river mouth was less than the CO₂ₑ added via the dolomite. Thus, additions of the carbonate alkaline amendment increased CO₂ emissions rather than drawing down CO₂. Comparison of the DIC above (KRC) and below the weathering zones (KRT in FIG. 6, following schema in FIG. 1E) showed that the presence of strong acids impaired carbonation weathering, thus releasing CO₂ [Equation (3)] instead of sequestering CO₂ [Equation (1)]. Further, the low pH in the river below the weathering zone also caused a loss of HCO₃⁻; while pH and HCO₃⁻ increased through the weathering zone, the increases were not maintained all the way to the river mouth (FIG. 8). Thus, because CDR was negative, the target was not met.

For Case B. CDR was found to be positive (FIG. 18C, Table 2), reaching the CDR target.

CDR for Case B can also be calculated from the amount of alkaline material added multiplied by carbonation weathering stoichiometry and efficiency factors [Equation (7)]. In this example summed for the study period, D=230 t dolomite, F=0.48, E=0.40, and G=1.6 tCO₂ₑ, resulting in a CDR_gross of 46 tCO₂ₑ and CDR_net of 44 tCO₂ₑ.

### Step G. Adjust Alkaline Amendment Method to Achieve Targets.

For Case A, because the CDR target was not met, the alkaline amendment application method was modified by adding a non-carbonate based alkaline amendment before the dolomite was added, neutralizing the strong acids allowing carbonation weathering of the dolomite, and increasing the alkalinity in the drainage waters leading from the addition sites to the river mouth. In addition to allowing carbonation weathering reactions to take place in the weathering zone, the addition of a non-carbonate-based alkaline amendment elevated the pH to above 6.3 through the drainage network to the river mouth allowing increased amounts of HCO₃⁻ to be delivered to the ocean, thus ensuring a continuous length of favorable water chemistry from the addition site to the ocean.

Other methods which can be used to increase CDR in acidic rivers with a negative CDR are: the introduction of a reactor where pCO₂ levels are elevated, using microbial catalysts for carbonation weathering, and increasing the application of dolomite rate to increase pH in weathering zone.

### Step H. Continue Monitoring Carbon and Water Variables and Calculating CDR. Re-Adjust Alkaline Amendment Addition Method if Needed.

As a result of the change in the alkaline amendment application method for Case A, monitoring of the treatment and baseline values showed that CDR was positive (Table 2 Case A Step H and FIG. 18B). Thus, with the adjustments in Step G, Case A had the CDR in the target zone (Step H in Table 1). Ongoing monitoring of baseline, treatment, and control data, and making adjustments to amendment method were performed so that CDR remains in target values over the long-term.

### Step I. Document Ecosystem Co-Benefits

Following the process outlined in FIG. 5, in Case A, environmental co-benefits of the process were determined. One ecological co-benefit was increasing pH above 6.5 from a previous value of 4.7, so that pH levels meet water quality standards. Another ecological co-benefit was the reduction toxic ionic aluminum to below toxic threshold levels. Another ecological co-benefit was the increased calcium concentrations to meet water quality levels. Both Case A and Case B will also have the ecological co-benefit of contributing to reduced ocean acidification by adding alkalinity to coastal zones (FIG. 19).

### Data Used to Demonstrate Methods

The following data were modelled for a 43-day period based on observations for Case A and Case B. Here the treatment monitoring site in all cases was at the river mouth.

CDR(DL) represents daily gross carbon dioxide removal (not including grey emissions). CDR(CLT) represents cumulative daily gross carbon dioxide removal through the study period. C represents the difference in bicarbonate flux at the river mouth [following Equation (5)]. I represents the amount of carbon input in the alkaline amendment (in tonnes CO₂ₑ). HCO₃₋ₜ represents the bicarbonate concentration at the treatment monitoring site during the study period in μmol/kg [following Equation (5)]. HCO_{3-b} represents the bicarbonate concentration for baseline conditions at the treatment monitoring site during the study period in μmol/kg. Baseline conditions were modelled from control, following the example method in FIG. 20, and following Equation (5). pHₜ represents the pH at the treatment monitoring site during the study period. pH_{b} represents the pH for baseline conditions at the treatment monitoring site during the study period. Baseline conditions were modelled from control following the example method in FIG. 20. Q represents the river discharge at the river mouth during the study period, in m³/day [following Equation (5)]. D represents the mass of alkaline amendment added in kg/day. Here only the mass of the dolomitic material was included. Mass of the non-carbonate based alkaline amendment used in Case A step H was not included in this total. gₐₐ is the prescribed dose of alkaline material per unit river discharge; here in this ratio only the dolomitic material was included.

TABLE 3A
Case A data for initial alkaline material addition method (Step F in FIG. 1A).

| | CDR (DL) | CDR (CLT) | C | I | HCO3-t | HCO3-b | | | Q | D | gaa |
|---|---|---|---|---|---|---|---|---|---|---|---|
| Date | t-CO2e | t-CO2e | t-CO2e | t-CO2e | μmol/kg | μmol/kg | pH-t | pH-b | m3/day | kg/day | mg/m3 |
| 16 Jun. 2022 | −0.17 | −0.17 | 0.06 | 0.23 | 7.4 | 1.1 | 4.8 | 4.7 | 223,336 | 500 | 2240 |
| 17 Jun. 2022 | −0.19 | −0.36 | 0.04 | 0.23 | 7.4 | 1.5 | 4.8 | 4.7 | 163,861 | 500 | 3050 |
| 18 Jun. 2022 | −0.01 | −0.37 | 0.03 | 0.23 | 7.4 | 1.9 | 4.8 | 4.7 | 137,331 | 500 | 3640 |
| 19 Jun. 2022 | −0.2 | −0.56 | 0.03 | 0.23 | 7.4 | 1.7 | 4.8 | 4.7 | 123,236 | 500 | 4060 |
| 20 Jun. 2022 | −0.18 | −0.75 | 0.05 | 0.23 | 7.4 | 1.3 | 4.8 | 4.7 | 170,208 | 500 | 2940 |
| 21 Jun. 2022 | −0.12 | −0.87 | 0.11 | 0.23 | 7.3 | 1.2 | 4.8 | 4.7 | 423,594 | 500 | 1180 |
| 22 Jun. 2022 | −0.16 | −1.03 | 0.07 | 0.23 | 7.3 | 1.5 | 4.8 | 4.7 | 263,599 | 500 | 1900 |
| 23 Jun. 2022 | −0.18 | −1.21 | 0.05 | 0.23 | 7.3 | 1.3 | 4.8 | 4.7 | 201,647 | 500 | 2480 |
| 24 Jun. 2022 | −0.18 | −1.38 | 0.05 | 0.23 | 7.3 | 1.3 | 4.8 | 4.7 | 201,647 | 500 | 2480 |
| 25 Jun. 2022 | −0.2 | −1.58 | 0.03 | 0.23 | 7.3 | 1.4 | 4.8 | 4.7 | 127,047 | 500 | 3940 |
| 26 Jun. 2022 | −0.2 | −1.78 | 0.03 | 0.23 | 7.2 | 1.5 | 4.8 | 4.7 | 115,685 | 500 | 4320 |
| 27 Jun. 2022 | −0.2 | −1.98 | 0.03 | 0.23 | 7.2 | 1.9 | 4.8 | 4.7 | 117,504 | 500 | 4260 |
| 28 Jun. 2022 | −0.2 | −2.18 | 0.03 | 0.23 | 7.2 | 2.1 | 4.8 | 4.7 | 128,382 | 500 | 3890 |
| 29 Jun. 2022 | −0.2 | −2.38 | 0.03 | 0.23 | 7.3 | 2.3 | 4.8 | 4.7 | 145,794 | 500 | 3430 |
| 30 Jun. 2022 | −0.15 | −2.53 | 0.05 | 0.23 | 7.2 | 1.9 | 4.8 | 4.7 | 195,940 | 500 | 2550 |
| 1 Jul. 2022 | −0.19 | −2.72 | 0.04 | 0.23 | 7.2 | 1.7 | 4.8 | 4.7 | 176,341 | 500 | 2840 |
| 2 Jul. 2022 | −0.1 | −2.82 | 0.13 | 0.23 | 7.2 | 1.9 | 4.8 | 4.7 | 561,906 | 500 | 890 |
| 3 Jul. 2022 | −0.1 | −2.91 | 0.13 | 0.23 | 7.2 | 1.8 | 4.8 | 4.7 | 565,260 | 500 | 880 |
| 4 Jul. 2022 | −0.19 | −3.1 | 0.04 | 0.23 | 7.2 | 1.8 | 4.8 | 4.7 | 175,392 | 500 | 2850 |
| 5 Jul. 2022 | −0.19 | −3.29 | 0.04 | 0.23 | 7.2 | 2 | 4.8 | 4.7 | 186,624 | 500 | 2680 |
| 6 Jul. 2022 | −0.19 | −3.48 | 0.04 | 0.23 | 7.1 | 2.2 | 4.8 | 4.7 | 175,309 | 500 | 2850 |
| 7 Jul. 2022 | −0.2 | −3.68 | 0.03 | 0.23 | 7.2 | 2.2 | 4.8 | 4.7 | 134,784 | 500 | 3710 |
| 8 Jul. 2022 | −0.2 | −3.88 | 0.03 | 0.23 | 7.2 | 2.6 | 4.8 | 4.7 | 172,862 | 500 | 2890 |
| 9 Jul. 2022 | −0.15 | −4.02 | 0.08 | 0.23 | 7.1 | 2.5 | 4.8 | 4.7 | 411,684 | 500 | 1210 |
| 10 Jul. 2022 | −0.16 | −4.18 | 0.07 | 0.23 | 7.1 | 2.5 | 4.8 | 4.7 | 330,098 | 500 | 1510 |
| 11 Jul. 2022 | −0.19 | −4.38 | 0.04 | 0.23 | 7.1 | 2.4 | 4.8 | 4.7 | 192,906 | 500 | 2590 |
| 12 Jul. 2022 | −0.19 | −4.57 | 0.04 | 0.23 | 7.1 | 1.5 | 4.8 | 4.7 | 147,254 | 500 | 3400 |
| 13 Jul. 2022 | −0.18 | −4.75 | 0.05 | 0.23 | 7.1 | 1.4 | 4.8 | 4.7 | 207,668 | 500 | 2410 |
| 14 Jul. 2022 | −0.19 | −4.93 | 0.04 | 0.23 | 7.0 | 1.7 | 4.8 | 4.7 | 190,380 | 500 | 2630 |
| 15 Jul. 2022 | −0.2 | −5.13 | 0.03 | 0.23 | 7.0 | 1.9 | 4.8 | 4.7 | 148,843 | 500 | 3360 |
| 16 Jul. 2022 | −0.21 | −5.34 | 0.02 | 0.23 | 7.1 | 1.9 | 4.8 | 4.7 | 94,919 | 500 | 5270 |
| 17 Jul. 2022 | −0.21 | −5.55 | 0.02 | 0.23 | 7.1 | 2.2 | 4.8 | 4.7 | 94,704 | 500 | 5280 |
| 18 Jul. 2022 | −0.22 | −5.76 | 0.01 | 0.23 | 7.0 | 2.3 | 4.8 | 4.7 | 63,391 | 500 | 7890 |
| 19 Jul. 2022 | −0.22 | −5.98 | 0.01 | 0.23 | 7.0 | 2.4 | 4.8 | 4.7 | 62,822 | 500 | 7960 |
| 20 Jul. 2022 | −0.2 | −6.18 | 0.03 | 0.23 | 7.1 | 1.9 | 4.8 | 4.7 | 123,302 | 500 | 4060 |
| 21 Jul. 2022 | −0.18 | −6.36 | 0.05 | 0.23 | 7.2 | 1.8 | 4.8 | 4.7 | 218,342 | 500 | 2290 |
| 22 Jul. 2022 | −0.19 | −6.55 | 0.04 | 0.23 | 7.2 | 1.8 | 4.8 | 4.7 | 172,800 | 500 | 2890 |
| 23 Jul. 2022 | −0.2 | −6.74 | 0.03 | 0.23 | 7.2 | 2.1 | 4.8 | 4.7 | 149,277 | 500 | 3350 |
| 24 Jul. 2022 | −0.2 | −6.94 | 0.03 | 0.23 | 7.1 | 2.4 | 4.8 | 4.7 | 143,741 | 500 | 3480 |
| 25 Jul. 2022 | −0.2 | −7.14 | 0.03 | 0.23 | 7.1 | 1.8 | 4.8 | 4.7 | 151,286 | 500 | 3300 |
| 26 Jul. 2022 | −0.16 | −7.3 | 0.07 | 0.23 | 7.1 | 1.9 | 4.8 | 4.7 | 317,862 | 500 | 1570 |
| 27 Jul. 2022 | −0.13 | −7.43 | 0.1 | 0.23 | 7.1 | 1.9 | 4.8 | 4.7 | 423,391 | 500 | 1180 |
| 28 Jul. 2022 | −0.15 | −7.58 | 0.08 | 0.23 | 7.2 | 2 | 4.8 | 4.7 | 368,091 | 500 | 1360 |
| 29 Jul. 2022 | −0.17 | −7.74 | 0.06 | 0.23 | 7.3 | 2.3 | 4.8 | 4.7 | 291,724 | 500 | 1710 |
| 30 Jul. 2022 | −0.17 | −7.91 | 0.06 | 0.23 | 7.3 | 2.4 | 4.8 | 4.7 | 269,018 | 500 | 1860 |
| 31 Jul. 2022 | −0.18 | −8.1 | 0.05 | 0.23 | 7.4 | 2.5 | 4.8 | 4.7 | 224,392 | 500 | 2230 |

TABLE 3B
Case A data for modified alkaline material addition method, where an additional
non-carbonate alkaline material was added to the process (Step H in FIG. 1A).

| | CDR (DL) | CDR (CLT) | C | I | HCO3-t | HCO3-b | | | Q | D | gaa |
|---|---|---|---|---|---|---|---|---|---|---|---|
| Date | t-CO2e | t-CO2e | t-CO2e | t-CO2e | μmol/kg | μmol/kg | pH-t | pH-b | m3/day | kg/day | mg/m3 |
| 16 Jun. 2022 | 1.84 | 1.84 | 2.07 | 0.23 | 210.7 | 0 | 6.71 | 4.67 | 223336 | 500 | 2239 |
| 17 Jun. 2022 | 1.33 | 3.18 | 1.56 | 0.23 | 216.4 | 0 | 6.73 | 4.67 | 163861 | 500 | 3051 |
| 18 Jun. 2022 | 0.73 | 3.91 | 0.96 | 0.23 | 158.5 | 0 | 6.59 | 4.67 | 137331 | 500 | 3641 |
| 19 Jun. 2022 | 0.98 | 4.89 | 1.21 | 0.23 | 222.7 | 0 | 6.75 | 4.67 | 123236 | 500 | 4057 |
| 20 Jun. 2022 | 1.2 | 6.09 | 1.43 | 0.23 | 190.6 | 0 | 6.69 | 4.67 | 170208 | 500 | 2938 |
| 21 Jun. 2022 | 2.8 | 8.89 | 3.02 | 0.23 | 162.1 | 0 | 6.63 | 4.67 | 423594 | 500 | 1180 |
| 22 Jun. 2022 | 1.8 | 10.69 | 2.02 | 0.23 | 174.4 | 0 | 6.68 | 4.68 | 263599 | 500 | 1897 |
| 23 Jun. 2022 | 1.54 | 12.23 | 1.77 | 0.23 | 199.5 | 0 | 6.75 | 4.69 | 201647 | 500 | 2480 |
| 24 Jun. 2022 | 1.15 | 13.38 | 1.38 | 0.23 | 155.3 | 0 | 6.67 | 4.69 | 201647 | 500 | 2480 |
| 25 Jun. 2022 | 0.57 | 13.95 | 0.8 | 0.23 | 142.7 | 0 | 6.65 | 4.69 | 127047 | 500 | 3936 |
| 26 Jun. 2022 | 0.48 | 14.43 | 0.7 | 0.23 | 138.1 | 0 | 6.65 | 4.69 | 115685 | 500 | 4322 |
| 27 Jun. 2022 | 0.72 | 15.15 | 0.95 | 0.23 | 183.1 | 0 | 6.79 | 4.69 | 117504 | 500 | 4255 |
| 28 Jun. 2022 | 0.57 | 15.72 | 0.79 | 0.23 | 140.6 | 0 | 6.67 | 4.71 | 128382 | 500 | 3895 |
| 29 Jun. 2022 | 0.5 | 16.21 | 0.72 | 0.23 | 112.5 | 0 | 6.58 | 4.7 | 145794 | 500 | 3429 |
| 30 Jun. 2022 | 1.26 | 17.47 | 1.48 | 0.23 | 172 | 0 | 6.76 | 4.7 | 195940 | 500 | 2552 |
| 1 Jul. 2022 | 0.79 | 18.26 | 1.02 | 0.23 | 131.2 | 0 | 6.62 | 4.7 | 176341 | 500 | 2835 |
| 2 Jul. 2022 | 3.1 | 21.36 | 3.32 | 0.23 | 134.4 | 0 | 6.63 | 4.69 | 561906 | 500 | 890 |
| 3 Jul. 2022 | 3.67 | 25.03 | 3.9 | 0.23 | 156.8 | 0 | 6.69 | 4.69 | 565260 | 500 | 885 |
| 4 Jul. 2022 | 1.22 | 26.25 | 1.45 | 0.23 | 187.4 | 0 | 6.77 | 4.69 | 175392 | 500 | 2851 |
| 5 Jul. 2022 | 0.8 | 27.05 | 1.02 | 0.23 | 124.4 | 0 | 6.59 | 4.69 | 186624 | 500 | 2679 |
| 6 Jul. 2022 | 1.06 | 28.11 | 1.29 | 0.23 | 167.4 | 0 | 6.73 | 4.69 | 175309 | 500 | 2852 |
| 7 Jul. 2022 | 0.65 | 28.76 | 0.87 | 0.23 | 147.1 | 0 | 6.67 | 4.69 | 134784 | 500 | 3710 |
| 8 Jul. 2022 | 0.81 | 29.57 | 1.04 | 0.23 | 136.9 | 0 | 6.65 | 4.69 | 172862 | 500 | 2892 |
| 9 Jul. 2022 | 2.26 | 31.83 | 2.49 | 0.23 | 137.3 | 0 | 6.67 | 4.69 | 411684 | 500 | 1215 |
| 10 Jul. 2022 | 1.92 | 33.76 | 2.15 | 0.23 | 148.1 | 0 | 6.72 | 4.68 | 330098 | 500 | 1515 |
| 11 Jul. 2022 | 1.24 | 35 | 1.47 | 0.23 | 172.9 | 0 | 6.79 | 4.69 | 192906 | 500 | 2592 |
| 12 Jul. 2022 | 0.64 | 35.65 | 0.87 | 0.23 | 134.4 | 0 | 6.69 | 4.69 | 147254 | 500 | 3395 |
| 13 Jul. 2022 | 0.66 | 36.31 | 0.89 | 0.23 | 97.1 | 0 | 6.54 | 4.68 | 207668 | 500 | 2408 |
| 14 Jul. 2022 | 0.9 | 37.2 | 1.12 | 0.23 | 133.9 | 0 | 6.71 | 4.69 | 190380 | 500 | 2626 |
| 15 Jul. 2022 | 0.51 | 37.71 | 0.74 | 0.23 | 112.6 | 0 | 6.64 | 4.68 | 148843 | 500 | 3359 |
| 16 Jul. 2022 | 0.16 | 37.87 | 0.38 | 0.23 | 92 | 0 | 6.56 | 4.7 | 94919 | 500 | 5268 |
| 17 Jul. 2022 | 0.33 | 38.2 | 0.55 | 0.23 | 132.7 | 0 | 6.74 | 4.7 | 94704 | 500 | 5280 |
| 18 Jul. 2022 | 0.13 | 38.33 | 0.36 | 0.23 | 128.7 | 0 | 6.75 | 4.71 | 63391 | 500 | 7888 |
| 19 Jul. 2022 | 0.15 | 38.48 | 0.37 | 0.23 | 135.2 | 0 | 6.81 | 4.73 | 62822 | 500 | 7959 |
| 20 Jul. 2022 | 0.21 | 38.68 | 0.43 | 0.23 | 79.8 | 0 | 6.61 | 4.73 | 123302 | 500 | 4055 |
| 21 Jul. 2022 | 0.71 | 39.39 | 0.93 | 0.23 | 97 | 0 | 6.72 | 4.72 | 218342 | 500 | 2290 |
| 22 Jul. 2022 | 0.68 | 40.07 | 0.91 | 0.23 | 119.8 | 0 | 6.84 | 4.74 | 172800 | 500 | 2894 |
| 23 Jul. 2022 | 0.44 | 40.51 | 0.67 | 0.23 | 101.7 | 0 | 6.8 | 4.74 | 149277 | 500 | 3349 |
| 24 Jul. 2022 | 0.36 | 40.87 | 0.58 | 0.23 | 92.1 | 0 | 6.74 | 4.74 | 143741 | 500 | 3478 |
| 25 Jul. 2022 | 0.44 | 41.31 | 0.66 | 0.23 | 99.7 | 0 | 6.78 | 4.74 | 151286 | 500 | 3305 |
| 26 Jul. 2022 | 0.88 | 42.19 | 1.11 | 0.23 | 79.1 | 0 | 6.64 | 4.74 | 317862 | 500 | 1573 |
| 27 Jul. 2022 | 1.42 | 43.61 | 1.65 | 0.23 | 88.4 | 0 | 6.65 | 4.73 | 423391 | 500 | 1181 |
| 28 Jul. 2022 | 2 | 45.61 | 2.23 | 0.23 | 137.4 | 0 | 6.86 | 4.74 | 368091 | 500 | 1358 |
| 29 Jul. 2022 | 1.52 | 47.12 | 1.74 | 0.23 | 135.8 | 0 | 6.83 | 4.73 | 291724 | 500 | 1714 |
| 30 Jul. 2022 | 1.3 | 48.42 | 1.52 | 0.23 | 128.7 | 0 | 6.77 | 4.73 | 269018 | 500 | 1859 |
| 31 Jul. 2022 | 0.87 | 49.29 | 1.1 | 0.23 | 110.9 | 0 | 6.68 | 4.72 | 224392 | 500 | 2228 |

TABLE 3C
Case B data for the sold alkaline material addition method (Step F in FIG. 1A).

| | CDR (DL) | CDR (CLT) | C | I | HCO3-t | HCO3-b | | | Q | D | gaa |
|---|---|---|---|---|---|---|---|---|---|---|---|
| Date | t-CO2e | t-CO2e | t-CO2e | t-CO2e | μmol/kg | μmol/kg | pH-t | pH-b | m3/day | kg/day | mg/m3 |
| 16 Jun. 2022 | 1.34 | 1.34 | 3.64 | 2.3 | 137.6 | 102.1 | 7.52 | 7.47 | 2333360 | 5000 | 2143 |
| 17 Jun. 2022 | 1.38 | 2.72 | 3.68 | 2.3 | 152.8 | 104.8 | 7.54 | 7.47 | 1738610 | 5000 | 2876 |
| 18 Jun. 2022 | 0.96 | 3.68 | 3.26 | 2.3 | 156.9 | 101.3 | 7.52 | 7.47 | 1333310 | 5000 | 3750 |
| 19 Jun. 2022 | 0.94 | 4.62 | 3.24 | 2.3 | 160.8 | 103.4 | 7.53 | 7.47 | 1282360 | 5000 | 3899 |
| 20 Jun. 2022 | 1.31 | 5.93 | 3.61 | 2.3 | 147.5 | 100.4 | 7.52 | 7.47 | 1742080 | 5000 | 2870 |
| 21 Jun. 2022 | 1.74 | 7.67 | 4.04 | 2.3 | 126.0 | 104.5 | 7.53 | 7.47 | 4275940 | 5000 | 1169 |
| 22 Jun. 2022 | 1.25 | 8.92 | 3.55 | 2.3 | 134.5 | 104.4 | 7.53 | 7.48 | 2685990 | 5000 | 1862 |
| 23 Jun. 2022 | 0.43 | 9.36 | 2.73 | 2.3 | 135.2 | 104.9 | 7.54 | 7.49 | 2046470 | 5000 | 2443 |
| 24 Jun. 2022 | 1.15 | 10.51 | 3.45 | 2.3 | 141.2 | 102.7 | 7.53 | 7.49 | 2036470 | 5000 | 2455 |
| 25 Jun. 2022 | 0.61 | 11.12 | 2.91 | 2.3 | 152.1 | 102.4 | 7.53 | 7.49 | 1330470 | 5000 | 3758 |
| 26 Jun. 2022 | 0.82 | 11.94 | 3.12 | 2.3 | 166.1 | 104.2 | 7.53 | 7.49 | 1146850 | 5000 | 4360 |
| 27 Jun. 2022 | 0.59 | 12.54 | 2.89 | 2.3 | 159.2 | 104.2 | 7.53 | 7.49 | 1195040 | 5000 | 4184 |
| 28 Jun. 2022 | 0.62 | 13.16 | 2.92 | 2.3 | 154.7 | 104.5 | 7.53 | 7.51 | 1323820 | 5000 | 3777 |
| 29 Jun. 2022 | 0.89 | 14.05 | 3.19 | 2.3 | 154.3 | 103.9 | 7.53 | 7.5 | 1437940 | 5000 | 3477 |
| 30 Jun. 2022 | 1.54 | 15.59 | 3.84 | 2.3 | 147.2 | 102.6 | 7.53 | 7.5 | 1959400 | 5000 | 2552 |
| 1 Jul. 2022 | 0.58 | 16.17 | 2.88 | 2.3 | 140.0 | 103.3 | 7.53 | 7.5 | 1783410 | 5000 | 2804 |
| 2 Jul. 2022 | 2.36 | 18.53 | 4.66 | 2.3 | 124.0 | 105.1 | 7.54 | 7.49 | 5589060 | 5000 | 895 |
| 3 Jul. 2022 | 2.26 | 20.79 | 4.56 | 2.3 | 117.7 | 99.4 | 7.51 | 7.49 | 5682600 | 5000 | 880 |
| 4 Jul. 2022 | 0.58 | 21.37 | 2.88 | 2.3 | 144.0 | 106.2 | 7.54 | 7.49 | 1733920 | 5000 | 2884 |
| 5 Jul. 2022 | 0.57 | 21.94 | 2.87 | 2.3 | 129.8 | 95.5 | 7.49 | 7.49 | 1896240 | 5000 | 2637 |
| 6 Jul. 2022 | 0.58 | 22.52 | 2.88 | 2.3 | 140.1 | 103.4 | 7.53 | 7.49 | 1783090 | 5000 | 2804 |
| 7 Jul. 2022 | 0.38 | 22.90 | 2.68 | 2.3 | 144.0 | 99.9 | 7.51 | 7.49 | 1377840 | 5000 | 3629 |
| 8 Jul. 2022 | 0.54 | 23.44 | 2.84 | 2.3 | 137.8 | 100.7 | 7.52 | 7.49 | 1738620 | 5000 | 2876 |
| 9 Jul. 2022 | 1.49 | 24.93 | 3.79 | 2.3 | 116.1 | 95.1 | 7.49 | 7.49 | 4096840 | 5000 | 1220 |
| 10 Jul. 2022 | 2.55 | 27.48 | 4.85 | 2.3 | 127.1 | 94.2 | 7.49 | 7.48 | 3350980 | 5000 | 1492 |
| 11 Jul. 2022 | 1.47 | 28.95 | 3.77 | 2.3 | 145.3 | 100.6 | 7.52 | 7.49 | 1919060 | 5000 | 2605 |
| 12 Jul. 2022 | 1.16 | 30.11 | 3.46 | 2.3 | 156.3 | 104.3 | 7.53 | 7.49 | 1512540 | 5000 | 3306 |
| 13 Jul. 2022 | 0.76 | 30.87 | 3.06 | 2.3 | 144.0 | 109.9 | 7.56 | 7.48 | 2036680 | 5000 | 2455 |
| 14 Jul. 2022 | 0.66 | 31.53 | 2.96 | 2.3 | 138.6 | 103.9 | 7.53 | 7.49 | 1933800 | 5000 | 2586 |
| 15 Jul. 2022 | 0.48 | 32.01 | 2.78 | 2.3 | 150.0 | 107.8 | 7.55 | 7.48 | 1498430 | 5000 | 3337 |
| 16 Jul. 2022 | 0.24 | 32.25 | 2.54 | 2.3 | 167.9 | 108.9 | 7.55 | 7.5 | 979190 | 5000 | 5106 |
| 17 Jul. 2022 | 0.24 | 32.49 | 2.54 | 2.3 | 171.6 | 111.3 | 7.56 | 7.5 | 957040 | 5000 | 5224 |
| 18 Jul. 2022 | 0.09 | 32.58 | 2.39 | 2.3 | 198.9 | 114.0 | 7.57 | 7.51 | 639910 | 5000 | 7814 |
| 19 Jul. 2022 | 0.11 | 32.69 | 2.41 | 2.3 | 199.2 | 116.1 | 7.58 | 7.53 | 658220 | 5000 | 7596 |
| 20 Jul. 2022 | 0.40 | 33.09 | 2.70 | 2.3 | 162.1 | 113.4 | 7.57 | 7.53 | 1263020 | 5000 | 3959 |
| 21 Jul. 2022 | 0.90 | 33.99 | 3.20 | 2.3 | 148.3 | 115.5 | 7.58 | 7.52 | 2213420 | 5000 | 2259 |
| 22 Jul. 2022 | 0.62 | 34.61 | 2.92 | 2.3 | 148.7 | 110.7 | 7.56 | 7.54 | 1748000 | 5000 | 2860 |
| 23 Jul. 2022 | 1.27 | 35.88 | 3.57 | 2.3 | 166.0 | 112.4 | 7.57 | 7.54 | 1512770 | 5000 | 3305 |
| 24 Jul. 2022 | 1.24 | 37.12 | 3.54 | 2.3 | 168.6 | 113.8 | 7.57 | 7.54 | 1467410 | 5000 | 3407 |
| 25 Jul. 2022 | 1.29 | 38.41 | 3.59 | 2.3 | 165.7 | 112.5 | 7.57 | 7.54 | 1532860 | 5000 | 3262 |
| 26 Jul. 2022 | 2.91 | 41.32 | 5.21 | 2.3 | 147.6 | 110.8 | 7.56 | 7.54 | 3218620 | 5000 | 1553 |
| 27 Jul. 2022 | 3.86 | 45.19 | 6.16 | 2.3 | 143.5 | 110.3 | 7.56 | 7.53 | 4213910 | 5000 | 1187 |
| 28 Jul. 2022 | 1.55 | 46.73 | 3.85 | 2.3 | 133.9 | 110.1 | 7.56 | 7.54 | 3660910 | 5000 | 1366 |
| 29 Jul. 2022 | 1.21 | 47.94 | 3.51 | 2.3 | 137.9 | 110.8 | 7.56 | 7.53 | 2947240 | 5000 | 1697 |
| 30 Jul. 2022 | 1.15 | 49.09 | 3.45 | 2.3 | 143.9 | 115.1 | 7.58 | 7.53 | 2720180 | 5000 | 1838 |
| 31 Jul. 2022 | 0.93 | 50.02 | 3.23 | 2.3 | 148.1 | 115.8 | 7.58 | 7.52 | 2273920 | 5000 | 2199 |

### Additional Detail on Methods

### Lime Dosers

Automated lime dosers using common programmable logistic controller (PLC) automation to monitor sensor data (e.g., water level, stream chemical conditions, supply levels, etc.) were used. Initial algorithms to interpret incoming data (e.g., estimating river discharge from water level data and determining appropriate dosing rate), and communicating operations to a centralize, offsite monitoring station were used, and then amended in Step H (FIG. 1A) to include CDR feedback.

### Monitoring of Instream Chemical Conditions

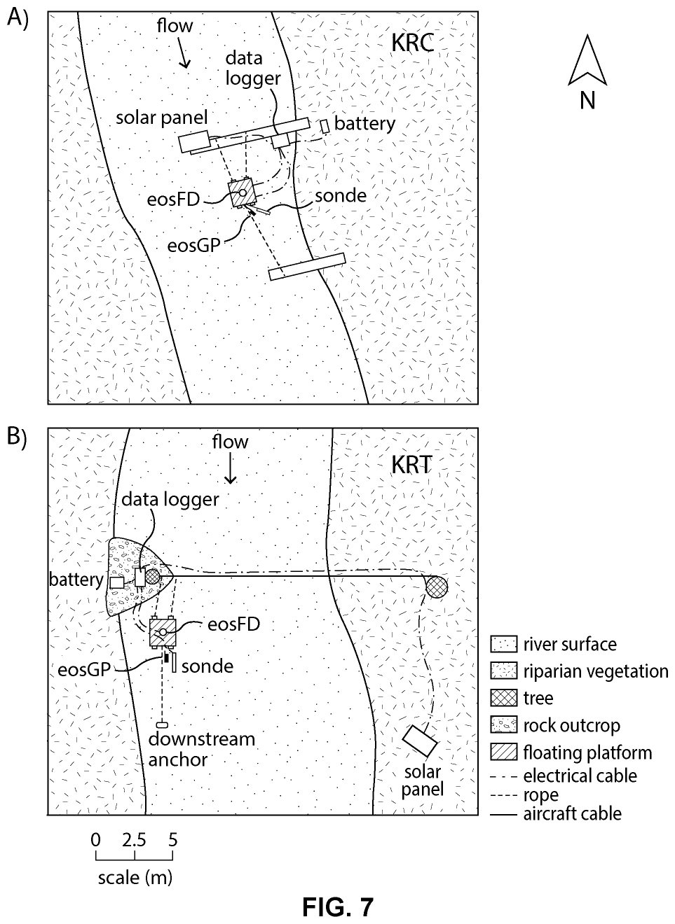

The monitoring of carbon and water variables needed to calculate CDR [discharge, pCO₂ or CO₂ concentration, temperature, conductivity and pH via Equation (4)], used water chemistry sensors that monitor these variables continuously (e.g., every 5 min or every hour) and are commercially available (FIG. 7).

A unique algorithm to determine the type, dose and frequency of material to be applied at each alkaline amendment addition site was developed, as a real-time function of water discharge, temperature, and chemistry. An iterative dosing method based on CDR calculations obtained by sensing CO₂ concentration, pH, temperature, and conductivity and discharge was used, so that CDR targets could be monitored, using Equations (4), (5) and (7). Dose amounts were recorded for CDR calculations in tonnes/day [variable D in Equation (7)]. Chemical concentration of the dolomite alkaline amendments was tracked. Discharge was measured in 15-minute increments to obtain mass flux calculations from concentration data [Equation (5)].

A method to add extra alkalinity in conditions where the pH is less than 4.5 was developed.

A method to add extra alkalinity in conditions where strong acids interfered with carbonation weathering was developed.

A method to add extra alkalinity in conditions where the pH is less than 6.3 was developed.

A method to add extra alkalinity in conditions where the pH is greater than 6.3 was developed.

The methods to add extra alkalinity are: 1) change the dosing algorithm to increase the amount of alkaline material added per unit time, 2) reduce loss of alkalinity by adding the alkaline reactant to the water in a reactor, where the water pH is raised to target levels before being released to the river, and 3) adding another addition point upstream of the existing addition point where non-carbonate mineral-based alkaline amendment were added to raise the pH and to remove the strong acids from solution.

Microbial amendment to increase carbonation weathering rates was used.

### Headwater Lake

When alkalinity was added directly to headwater lakes, the amount, timing and frequency of additions was based on the collection of detailed bathymetric data, limnological characterizations of each lake with specific attention to stratification schedules, and consideration of lake discharge rates. Access to lakes for logistics, such as emersion of a specialized liming barge or the delivery of alkaline material was a parameter taken into consideration.

### Monitoring Conditions

Temperature, pH, carbon variables, HCO₃⁻, pCO₂, and/or CO₂ concentrations downstream in a transect from the doser site down to the river mouth, or within the lake basin and downstream to the river mouth were monitored. The carbonate system was calculated from these measurements using CO₂SYS. When the conditions in the river at the doser were identified as being outside carbonation weathering conditions for the material being used, the river was treated with alkaline material. For example, for limestone: conditions should optimally be pH>4.5 and pCO₂>1.0 atm for carbonation weathering to occur, in the absence of strong acids. In Case A, Step F (FIG. 1A) for the initial alkaline material application method, it has been found that the presence of strong acids and low pH were causing CDR<0. In Step G (FIG. 1A) of Case A the alkaline material application method alteration was modified such that when the doser was used, a signal to upstream dosers was sent to increase the input of alkalinity by adding a non-carbonate based alkaline amendment to raise pH to >6.5 to avoid loss of CO₂ from the system allow carbonation weathering to occur when the dolomite was added at the second doser. The presence of strong acids is common in acidic rivers and those high proportion of peatlands/wetlands in the watershed, or in the presence of pyritic slate in the watershed (Lehmann et al., 2022).

This situation, where the conditions were outside carbonation weathering conditions may have occurred because of the presence of strong acids, flood conditions, or during snowpack melt events. Conditions where a reverse reaction (precipitation of CaCO₃) may occur (e.g., very high Ca²⁺ or HCO₃⁻ levels) were monitored. Concentrations of pCO₂ were monitored to ensure primary production was not limited by lowered CO₂ concentrations due to increased pH.

The following conditions were accounted for CO₂ drawdown: the mass of alkaline amendment applied and reaction stoichiometry [I in Equation (4)]; differences in treated and baseline carbon variables values; and pH of waters downstream of dosing site(s) to the ocean. Carbon drawdown was calculated using Equations (4), (5) and/or (7). Metals and water chemistry was monitored to ensure no unintended negative consequences occur.

### Field Measurement of Carbon Concentration and Carbon Fluxes

The experimental infrastructure consisted of control monitoring stations and treatment monitoring stations (FIG. 6). All stations measured carbon variables and water variables at control sites upstream of lime dosers and treatment sites downstream of lime dosers in two rivers (Killag and West) (Killag River is show in FIG. 6). The red star represents the addition site and the circles represent upstream and downstream monitoring sites (control and treatment sites, respectively). AF_WR is the river mouth.

The monitoring stations consisted of a CO₂ concentration sensor (eosGP unit) to measure CO₂ concentration, which is then converted to pCO₂ values, a pH and temperature sensor (HOBO MX2501 unit), a conductivity sensor, and an optional CO₂ Flux Sensor (eosFD unit) to measure CO₂ flux (FIG. 7). Continuous monitoring to collect data for the modelling took place from June 23 to Dec. 1, 2021. All stations were sited in calm-water areas to reduce signal noise associated with stream turbulence. CO₂SYS was used to calculate the values of variables in the carbonate system from the measured values.

Two roving stations were used for transects, and short and longer-term spot checks to determine the extent of acidification of rivers in the region. Roving stations were assemblages of sensors similar to that used in permanent stations but modified to permit transportation between sites. Modifications included the removal of anchors, a reduction in the size and buoyancy of floats or mounting of the equipment to the bow or gunnels of watercraft.

To map patterns of CO₂ flux downstream from the lime doser to estimate the carbon sequestration in the oceans, a transect of a 9 km reach down the Killag River to the ocean was conducted. On Sep. 8, 2021, an eosFD unit was carried downstream from just above the Killag River lime doser to a location approximately 9 kilometers downstream of the doser, collecting a 20-minute flux measurement every kilometer.

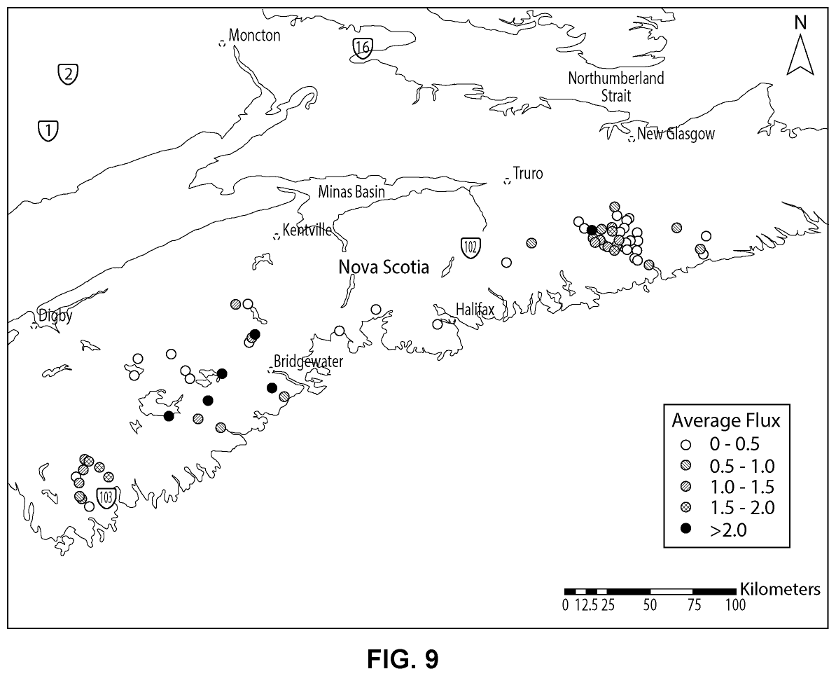

To further understand the range of carbon fluxes in untreated rivers in the region, CO₂ fluxes were measured at 84 short-term (30-60 minutes) and seven long-term (3-5 days) measurements with the eosFD unit at sites across Nova Scotia in September-November 2021. The average CO₂ flux ranged from −0.07 to 7.11 at sites outside the exemplary embodiments in Nova Scotia (FIG. 9, 16, 17).

### Stream Chemistry Sampling

During the experimental period, 20 grab sampling events took place at the control and treatment sites on the Killag River and 15 grab sampling events took place at the control and treatment sites on the West River. The collected samples were analyzed for water chemistry including alkalinity and partial pressure of carbon dioxide (pCO₂).

At each site visit, in-situ measurements of pH, water temperature, and specific conductance were collected side-by-side at the permanent stations, for quality analysis/quality assurance, using a handheld YSI Pro10 multiparameter water quality meter. This unit was calibrated immediately before sampling.

Grab samples to be analyzed for metals' content were collected using sterilized polyethylene syringes into sterilized polyethylene tubes (15 mL). These samples were filtered in the field and preserved with nitric acid (HNO₃) within a week of arriving at the laboratory. Dissolved metals were measured by passing the sample through a 0.45 um polyethersulfone (PES) filter. Organic metals were measured by passing a filtered sample through a 3 cm negatively charged cation exchange column (Bond Elut Jr. Strong Cation Exchange Column). Grab samples to be analyzed for water chemistry parameters including total organic carbon (TOC) and alkalinity were not filtered and were collected in sterilized amber glass bottles (1 L). All samples were kept cooled to a temperature of 4° C. during transport to the lab and were delivered to the lab within 48 hours of being collected.

Samples to be analyzed for pCO₂ were collected in triplicate using sterilized polyethylene syringes and stopcocks into sterilized glass headspace vials (20 mL). Vials were sealed with no headspace using a hand crimper and aluminum seals with flat butyl septa. Samples were preserved with mercuric chloride (HgCl₂) and kept cooled to a temperature of 4° C. prior to analysis.

Samples to be analyzed for alkalinity were collected in 250-mL glass media bottles. Samples were preserved with mercuric chloride (HgCl₂) and stored at room temperature prior to analysis.

### Results

Confirmation that the Addition of Alkalinity Reduces CO₂ Fluxes

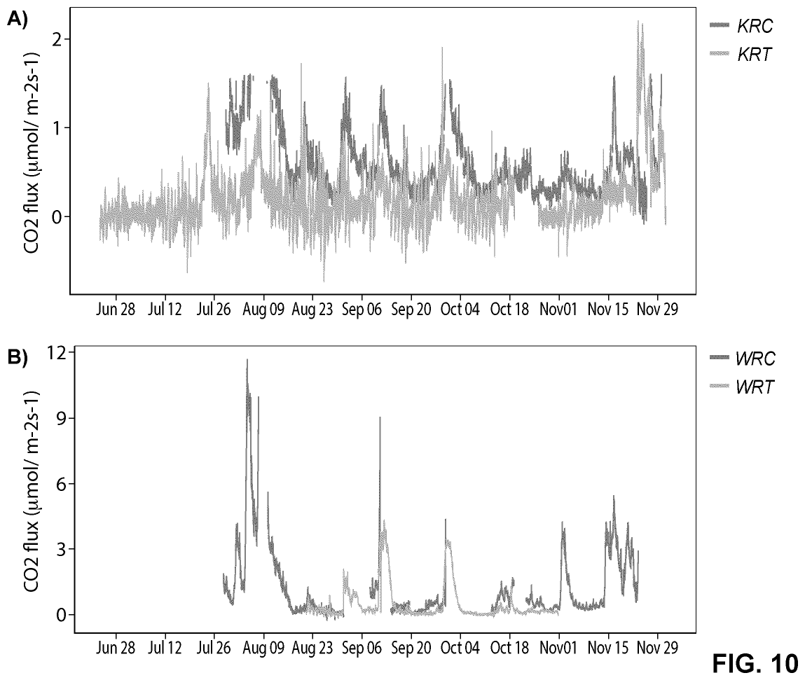

Field testing demonstrated that addition of alkalinity reduces CO₂ fluxes. CO₂ flux was significantly higher at the control sites (KRC and WRC) than the treatment sites (KRT and WRT) in both Killag and West rivers (FIGS. 10, 11, 12, Table 3).

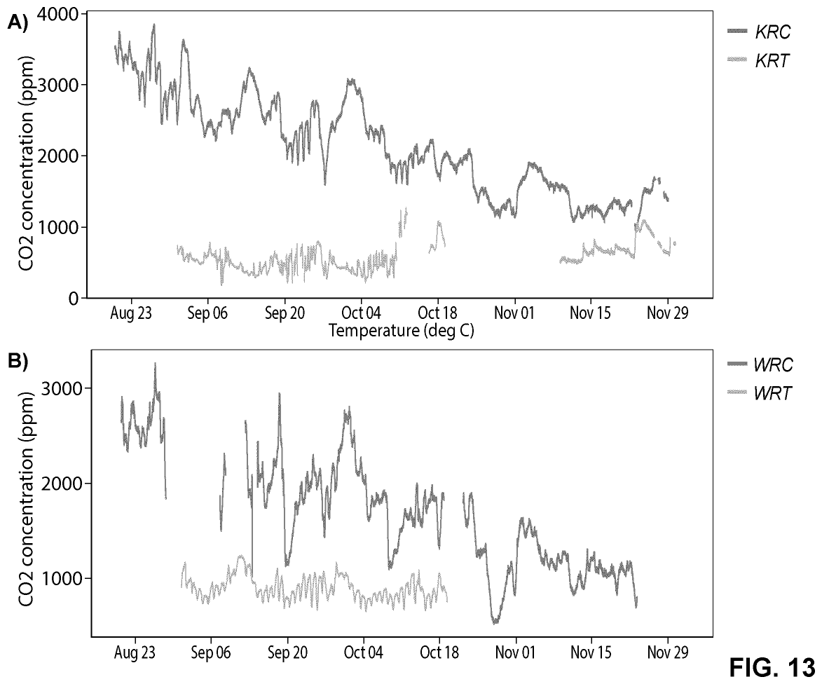

Field testing demonstrated that the addition of alkalinity reduces CO₂ concentration and pCO₂. CO₂ concentration was significantly higher at the control sites (KRC and WRC) than at the treatment sites (KRT and WRT) in both Killag and West rivers (FIGS. 13, 14, 15, Table 1).

TABLE 3
Median values for water chemistry parameters at control and treatment
sites for the 2021 measurement period. All differences between control
and treatment sites are statistically significant (p < 0.0001).

| | CO₂ flux | CO₂ concentration | |
|---|---|---|---|
| Site | (μm ol m−2 s−1) | (ppm) | pH |
| KRC | 0.46 | 2351 | 4.66 |
| KRT | 0.13 | 552 | 6.49 |
| WRC | 0.54 | 1628 | 4.32 |
| WRT | 0.16 | 879 | 6.36 |

### Environmental Conditions Altering CO₂ Flux

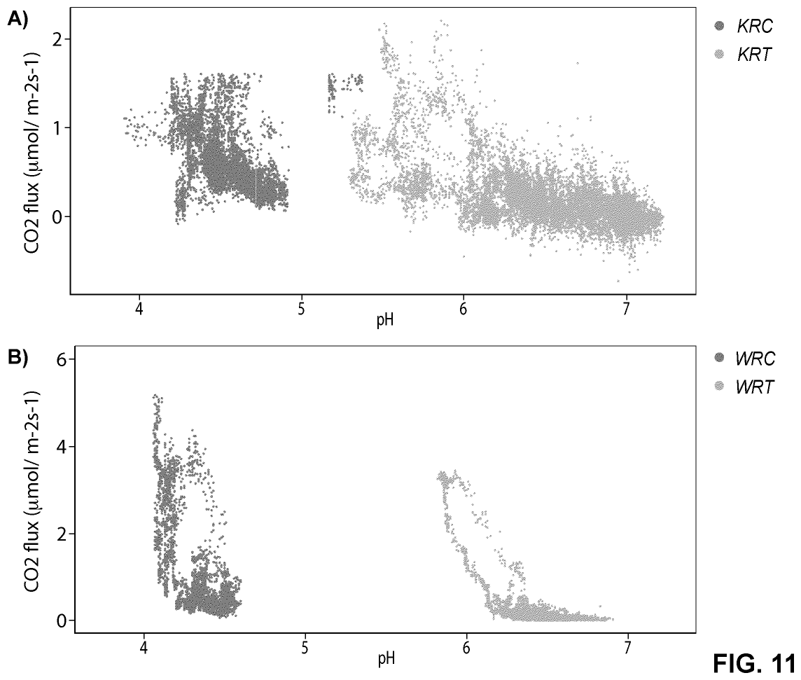

Decreasing pH was significantly correlated with increasing CO₂ flux at all permanent stations. The relationship was stronger at the control sites in both rivers (FIGS. 11A and 11B).

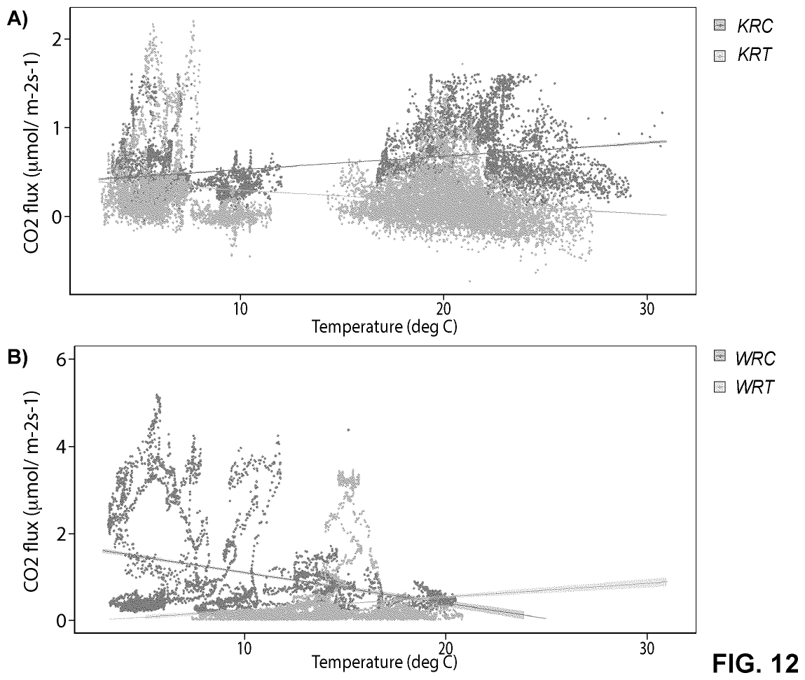

Increasing water temperature increased the difference in CO₂ flux between the control and treatment sites in the Killag river (FIG. 12A). In the West River the relationship was less clear, as lower temperatures had high spikes of CO₂ flux, which may be associated with flushing of cold water from the lake upstream during flood events (FIG. 12B).

### Effect of Liming on River CO₂ Concentrations

Liming significantly reduced aqueous CO₂ concentrations, which were significantly higher at the control sites than at the treatment sites (FIGS. 13A and 13B, Table 3).

Liming significantly increased HCO₃ concentrations, which were significantly higher downstream of the addition sites (FIG. 8).

### Effects of Environmental Parameters on CO₂ Concentration

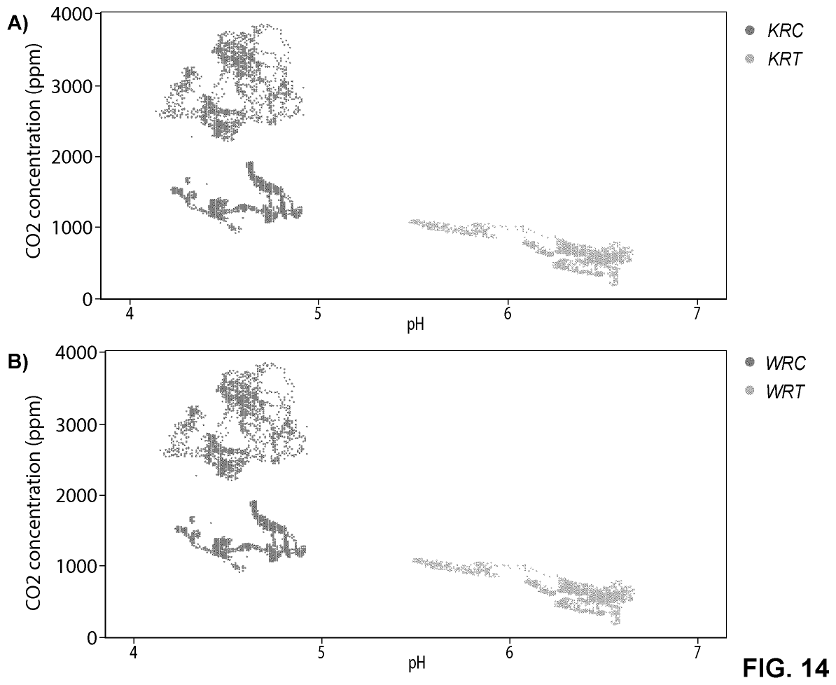

Increasing pH significantly reduced CO₂ concentration (FIGS. 14A and 14B), along the range of pH at the four sites.

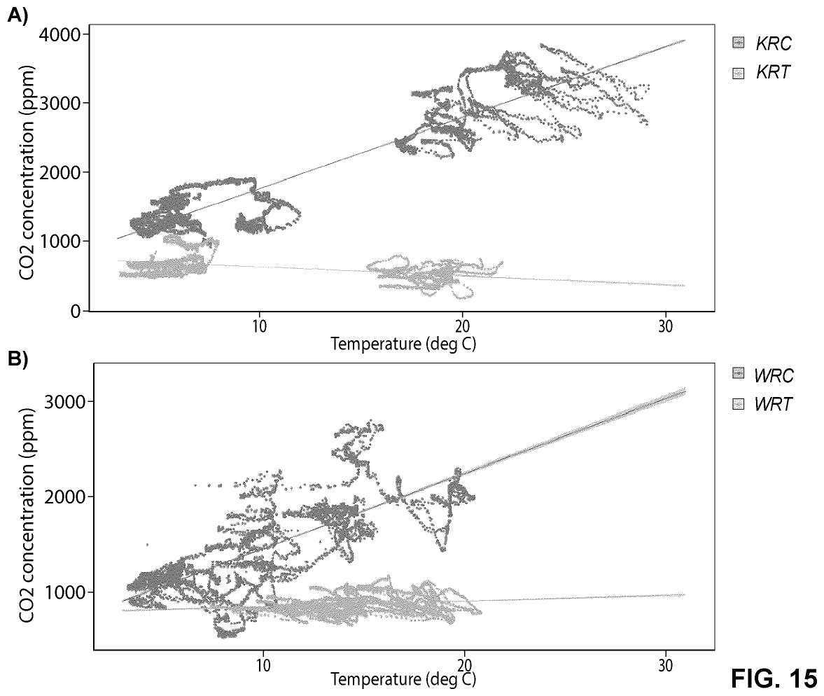

Increasing water temperature significantly increased aqueous CO₂ concentration at the untreated sites. The sites treated with liming do not have an increase in CO₂ flux with increasing temperature (FIGS. 15A and 15B).

### Patterns of CO₂ Flux in Untreated Rivers Across Acidified Areas of Nova Scotia

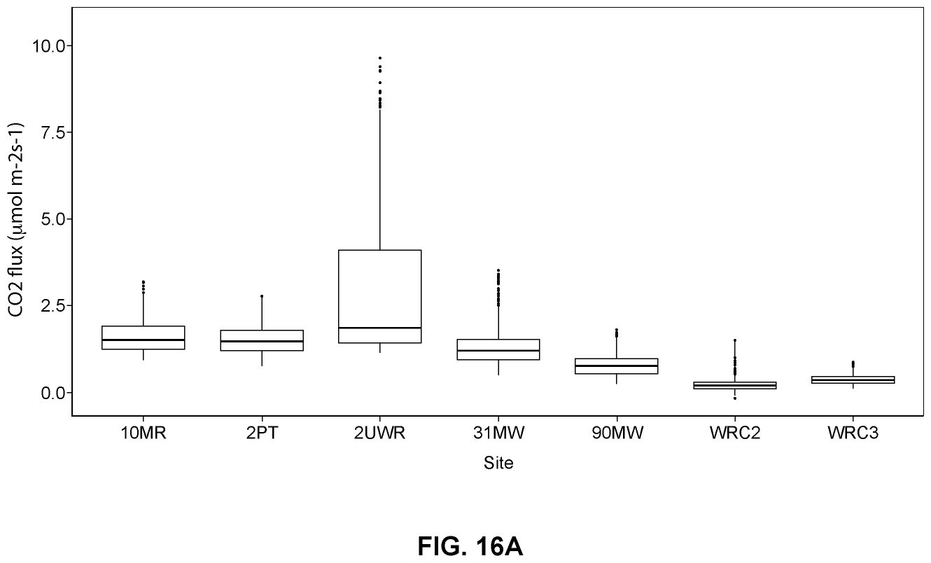

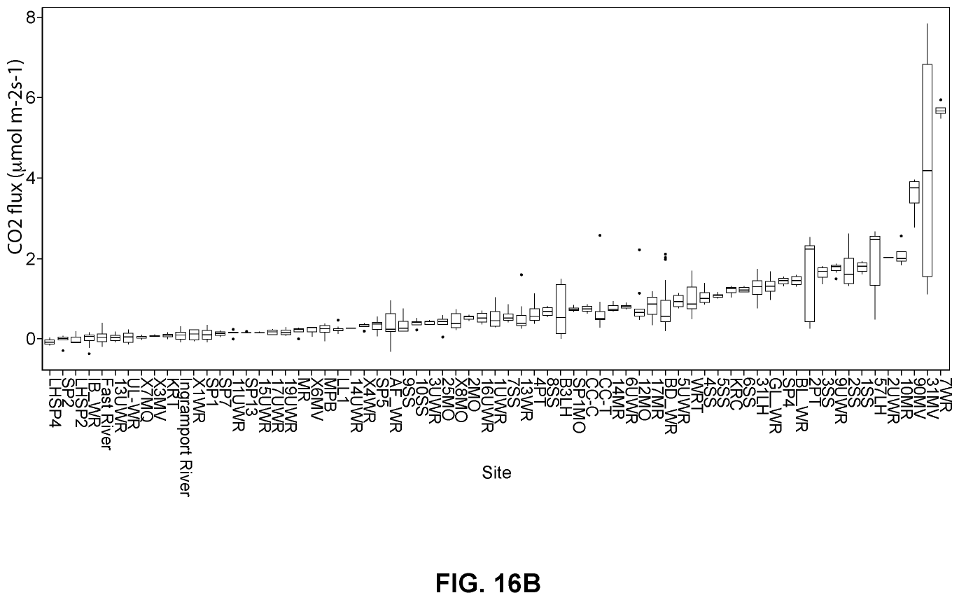

The average CO₂ flux ranged from 0.22 to 3.15 at longer-term float sites (FIG. 16B) and ranged from −0.07 to 7.11 at the spot-check float sites (FIG. 9, 16A). CO₂ flux was significantly higher with lower pH across the longer-term and spot check float sites (FIG. 17).

While the above description provides examples of one or more apparatuses or methods, it will be appreciated that other apparatuses or methods may be within the scope of the accompanying claims.

### FULL CITATIONS FOR DOCUMENTS REFERRED TO IN THE SPECIFICATION

Bach, L. T., Gill, S. J., Rickaby, R. E., Gore, S., & Renforth, P. 2019. CO2 removal with enhanced weathering and ocean alkalinity enhancement: potential risks and co-benefits for marine pelagic ecosystems. Frontiers in Climate, 1, 7.
Beerling, D. J., Leake, J. R., Long, S. P., Scholes, J. D., Ton, J., Nelson, P. N., Bird, M., Kantzas, E., Taylor, L. L. and Sarkar, B.: Farming with crops and rocks to address global climate, food and soil security, Nature plants, 1, 2018.
Beerling, D. J., Kantzas, E. P., Lomas, M. R., Wade, P., Eufrasio, R. M., Renforth, P., . . . & Banwart, S. A. 2020. Potential for large-scale CO 2 removal via enhanced rock weathering with croplands. Nature, 583(7815), 242-248.
Berner, Robert A., and Antonio C. Lasaga. Modeling the geochemical carbon cycle. Scientific American 260.3 (1989): 74-81.
Bertagni, Matteo B., and Amilcare Porporato. The Carbon-Capture Efficiency of Natural Water Alkalinization: Implications For Enhanced weathering. Science of The Total Environment 838 (2022): 156524.
Fawzy, S., Osman, A. I., Doran, J., & Rooney, D. W. 2020. Strategies for mitigation of climate change: a review. Environmental Chemistry Letters, 1-26.
Hamilton, S. K., Kurzman, A. L., Arango, C., Jin, L. and Robertson, G. P.: Evidence for carbon sequestration by agricultural liming, Global Biogeochem. Cycles, 21, 2007.
Jens Hartmann, A. Joshua West, Phil Renforth, Peter Köhler, Christina L. De La Rocha, Dieter A. Wolf-Gladrow, Hans H. Darr, and Jürgen, Enhanced chemical weathering as a geoengineering strategy to reduce atmospheric carbon dioxide, supply nutrients, and mitigate ocean acidification, Reviews of Geophysics, 51/2013.
Horgby, Å., Segatto, P. L., Bertuzzo, E., Lauerwald, R., Lehner, B., Ulseth, A. J., . . . & Battin, T. J. 2019. Unexpected large evasion fluxes of carbon dioxide from turbulent streams draining the world's mountains. Nature communications, 10(1), 1-9.
IPCC, 2018: Climate Change 2018: The Physical Science Basis. Contribution of Working Group I to the Fifth Assessment Report of the Intergovernmental Panel on Climate Change [Masson-Delmotte, Cambridge University Press.
IPCC, 2021: Climate Change 2021: The Physical Science Basis. Contribution of Working Group I to the Sixth Assessment Report of the Intergovernmental Panel on Climate Change [Masson-Delmotte, V., P. Zhai, A. Pirani, S. L. Connors, C. Péan, S. Berger, N. Caud, Y. Chen, L. Goldfarb, M. I. Gomis, M. Huang, K. Leitzell, E. Lonnoy, J. B. R. Matthews, T. K. Maycock, T. Waterfield, O. Yelekçi, R. Yu, and B. Zhou (eds.)]. Cambridge University Press. In Press.
Kantola, I. B., Masters, M. D., Beerling, D. J., Long, S. P. and DeLucia, E. H.: 2017. Potential of global croplands and bioenergy crops for climate change mitigation through deployment for enhanced weathering, Biology letters, 13, 20160714, 2017.
Kokic, J., M. B. Wallin, H. E. Chmiel, B. A. Denfeld, and S. Sobek (2015), Carbon dioxide evasion from headwater systems strongly contributes to the total export of carbon from a small boreal lake catchment, J. Geophys. Res. Biogeosci., 120, doi:10.1002/2014JG002706.
Lehmann, Nele, et al. Alkalinity generation from carbonate weathering in a silicate-dominated headwater catchment at Iskorasfjellet, northern Norway. Biogeosciences Discussions (2022): 1-39.
Lewis, E., and D. W. R. Wallace. CO2SYS-Program developed for the CO2 system calculations. Carbon Dioxide Inf Anal Center Report ORNL/CDIAC-105 (1998).
Middelburg, J. J., Soetaert, K., and Hagens, M. (2020), Understanding alkalinity to quantify ocean buffering, Eos, 101, https://doi.org/10.1029/2020EO146562. Published on 29 Jul. 2020.
Mook, W., and Kazimierz Rozanski. Environmental isotopes in the hydrological cycle. IAEA Publish 39 (2000). Chapter 9.
Mumma, H., et al., 2022, Continuous Monitoring of Net CO2 Drawdown from Experimental Ocean Alkalinity Additions, A G U, 2022.
Renforth, P., & Henderson, G. (2017). Assessing ocean alkalinity for carbon sequestration. Reviews of Geophysics, 55(3), 636-674.
Terlouw, T., Bauer, C., Rosa, L. and Mazzotti, M., 2021. Life cycle assessment of carbon dioxide removal technologies: a critical review. Energy & Environmental Science, 14(4), pp. 1701-1721.
Uroz, S., Calvaruso, C., Turpault, M. P., & Frey-Klett, P. (2009). Mineral weathering by bacteria: ecology, actors and mechanisms. Trends in microbiology, 17(8), 378-387.
Wallin, M. B., Grabs, T., Buffam, I., Laudon, H., Agren, A., Öquist, M. G., & Bishop, K. 2013. Evasion of CO₂ from streams—The dominant component of the carbon export through the aquatic conduit in a boreal landscape. Global Change Biology, 19(3), 785-797.

1. A method of removing atmospheric carbon dioxide (CO₂), the method comprising:
identifying a target inland waterbody having connectivity to an ocean;
selecting one or more alkaline materials to be used;
locating one or more addition sites in the target inland waterbody;
establishing pre-treatment alkalinity status at the one or more addition sites in the target inland waterbody to determine one or more methods of adding the one or more alkaline materials at the one or more addition sites; and
adding the one or more alkaline materials to the one or more addition sites in the target inland waterbody.

2. The method of claim 1, wherein the target inland waterbody is a pond, a stream, a lake, a river, a man-made channel or conduit, an estuary, or a wetland.

3. (canceled)

4. (canceled)

5. The method of claim 1, wherein the target inland waterbody comprises at least one of a temperature of about 0° C. to about 45° C., mean pH of about 6.3 to about 9.0, a Ca²⁺ concentration of more than about 5 mg/L, HCO₃₋ concentration of more than about 0.6 mM, a conductivity of more than about 50 uS/cm, or any combination thereof.

6. (canceled)

7. The method of claim 1, wherein the target inland waterbody comprises a drainage network of target inland waterbody watershed, or the target inland waterbody comprises a segment of a drainage network of the target inland waterbody watershed, and the segment of the drainage network is located between headwaters and a point of interest of the target inland waterbody watershed.

8. (canceled)

9. (canceled)

10. The method of claim 1, wherein the step of identifying the target inland waterbody comprises testing at least one carbon variable and at least one water variable of the target inland waterbody, and the step of identifying the target inland waterbody comprises testing pH and bicarbonate concentrations.

11-24. (canceled)

25. The method of claim 1, wherein the step of selecting one or more alkaline materials comprises selecting one or more alkaline materials from carbonate rocks comprising minerals such as dolomite, or calcite; igneous/metamorphic rocks rich in alkaline earth metals, such as basalt, containing such minerals as wollastonite, calcium-rich plagioclase feldspar, olivine, and calcium and magnesium-rich pyroxenes; industrially-produced alkaline material, such as cement kiln dust, magnesium or calcium oxides; industrial materials containing alkali metals or alkaline earth metals; or any combination thereof; and the median grain size of the one or more alkaline materials is between about 0.06 μm to about 64 mm.

26-32. (canceled)

33. The method of claim 1, further comprising locating one or more addition sites in the target inland waterbody, and the one or more addition sites are located adjacent to, within, above or below a dam, and/or the one or more addition sites are located adjacent to, within, or near industrial infrastructure selected from a storage silo, a reactor, a mine, a quarry and a cement plant.

34-40. (canceled)

41. The method of claim 1, wherein the pre-treatment alkalinity status is determined based on at least one of alkalinity, carbon variable, strong acids and pH values of the inland waterbodies that form the drainage network from the one or more addition sites to a river mouth or combinations thereof.

42-54. (canceled)

55. The method of claim 1, wherein the one or more methods of adding the one or more alkaline materials at the one or more addition sites comprises using alkaline amendment application infrastructure, and the alkaline amendment application infrastructure is selected from a doser, a reactor, a barge, a boat, a helicopter, a truck, a conveyor belt, an airplane and a drone.

56-61. (canceled)

62. The method of claim 55, wherein the alkaline amendment application infrastructure is the reactor, and the reactor comprises a flue gas link to add concentrated CO₂ to reaction that takes place in the reactor.

63. The method of claim 62, wherein the reactor is a two-staged reactor, and the reactor comprises one or more agitators.

64-66. (canceled)

67. The method of claim 1, wherein the step of adding the one or more alkaline materials further comprises establishing bicarbonate concentration at a point of interest and/or near a river mouth of the target inland waterbody watershed, and the bicarbonate concentration is established by direct measurement of one or more carbon variables, through modelling of the bicarbonate concentration, or through direct measurement of bicarbonate via sensor.

68-70. (canceled)

71. The method of claim 1, wherein the step of adding the one or more alkaline materials further comprises monitoring at one or more monitoring sites, and further comprising testing one or more of carbon variable, ecological variable, water variable and CDR values at the one or more monitoring sites to determine the response of the target inland waterbody and/or its inland waterbodies that connect the one or more addition sites of the one or more alkaline materials to a river mouth.

72-78. (canceled)

79. The method of claim 71, further comprising establishing target ranges of one or more of the carbon variable, ecological variable water variable and CDR values at the one or more monitoring sites, and further comprising adjusting the adding the one or more alkaline materials to achieve the values in the target ranges.

80. (canceled)

81. The method of claim 79, further comprising monitoring or estimating one or more of the carbon variable, ecological variable water variable and CDR values at the one or more monitoring sites.

82. The method of claim 79, further comprising calculating baseline values of the one or more of the carbon variable, ecological variable and water variable values at the one or more monitoring sites.

83-91. (canceled)

92. The method of claim 1, further comprising calculating carbon capture by estimating the amount of CO₂ removal from the addition of the one or more alkaline materials to the target inland waterbody, and the amount of CO₂ drawdown is estimated from increased carbonation weathering, increased alkalinity, or a combination thereof.

93. (canceled)

94. The method of claim 92, wherein the estimating the amount of CO₂ drawdown (CDR) is obtained from the one or more treatment sites, treatment monitoring sites, control sites, estimation of baseline values, or any combination thereof.

95. (canceled)

96. A method, comprising:
identifying a target inland waterbody having connectivity to an ocean;
selecting one or more alkaline materials to be used;
locating one or more addition sites in the target inland waterbody;
establishing pre-treatment alkalinity status at the one or more addition sites in the target inland waterbody to determine one or more methods of adding the one or more alkaline materials at the one or more addition sites; and
adding the one or more alkaline materials to the one or more addition sites in the target inland waterbody,
wherein the one or more methods of adding the one or more alkaline materials at the one or more addition sites comprises using alkaline amendment application infrastructure,
wherein the alkaline amendment application infrastructure comprises a reactor, and
wherein the reactor comprises a flue gas link to add concentrated CO₂ to a reaction that takes place in the reactor.

97. A method, comprising:
identifying a target inland waterbody having connectivity to an ocean;
selecting one or more alkaline materials to be used;
locating one or more addition sites in the target inland waterbody;
establishing pre-treatment alkalinity status at the one or more addition sites in the target inland waterbody to determine one or more methods of adding the one or more alkaline materials at the one or more addition sites;
adding the one or more alkaline materials to the one or more addition sites in the target inland waterbody; and
calculating carbon capture by estimating the amount of CO₂ removal from the addition of the one or more alkaline materials to the target inland waterbody,
wherein the amount of CO₂ drawdown is estimated from increased carbonation weathering, increased alkalinity, or a combination thereof, and
wherein the estimating the amount of CO₂ drawdown (CDR) is obtained from the one or more treatment sites, treatment monitoring sites, control sites, estimation of baseline values, or any combination thereof.
